(19) United States
(12) Patent Application Publication
(10) Pub. No.: US 2014/0296499 A1
(43) Pub. Date: Oct. 2, 2014

(54) Novel Process for the Preparation of Rebaudioside D and Other Related Naturally Occurring Sweetners

(71) Applicant: SU ZHOU JING HONG BIOTECH CO., LTD., Wujiang, Jiangsu Province (CN)

(72) Inventors: Ping Chen, Hillsborough, NJ (US); Yinqiang Li, Shanghai (CN); Shaoping Peng, Shanghai (CN)

(21) Appl. No.: 14/362,627

(22) PCT Filed: Dec. 10, 2012
(86) PCT No.: PCT/IB2012/003095

§ 371 Date: Jun. 4, 2014

Related U.S. Application Data
(60) Provisional application No. 61/579,016, filed on Dec. 22, 2011.

(30) Foreign Application Priority Data
Dec. 9, 2011 (CN) 201110408176.7

(51) Int. Cl. C07H 15/24

(52) U.S. Cl. CPC C07H 15/24
USPC 536/18.1

(57) ABSTRACT

A novel process for preparation of Rebaudioside D (RD), and other related naturally occurring sweeteners is provided. RD is a natural sweetening agent which can decrease the bitter aftertaste of steviol glycosides. The said process is suitable for commercial manufacturing by using readily available natural products and nontoxic reagents.

### CROSS REFERENCE TO RELATED APPLICATIONS

This application claims the benefit of Chinese Application No. 201110408176.7, filed Dec. 9, 2011, and U.S. Provisional Patent Application No. 61/579,016, filed Dec. 22, 2011, the entire contents of which are hereby incorporated by reference herein.

### FIELD OF THE INVENTION

The present invention relates to a novel process for preparation of Rebaudioside D (RD), and other related naturally occurring sweeteners (such as Rebaudioside M. Rebaudioside N, Rebaudioside I, Rebaudioside O, and etc.). RD is a natural sweetening agent which can decrease the bitter aftertaste of steviol glycosides. The invention relates to an efficient process that is suitable for commercial manufacturing by using readily available natural products and nontoxic reagents.

### BACKGROUND OF THE INVENTION

Steviol glycosides are considered high intensity sweeteners and have been used for several years in a number of countries as a sweetener for a range of food products. The aqueous solutions of Steviol glycoside preparations are 200 to 300 times sweeter than sucrose under identical conditions. With this significant advantage, Stevia has garnered attention with the rise in demand for low carbohydrate, low sugar food alternatives.

Among the different kind of Steviol glycosides, Stevioside (ST) and Rebaudioside A (RA) are the principal sweetening compounds and generally accompanied by smaller amounts of other Steviol glycosides. Both of them have slower onset and longer duration than that of sugar, and are deemed very close to sucrose. Unfortunately, compositions that contain Stevioside and Rebaudioside A (RA) typically have a bitter aftertaste at high concentration which greatly limited their application. It has been found that by increasing the amount of Rebaudioside D (another Steviol glycosides in Stevia) in a composition that includes rebaudioside A and/or other stevia components can greatly overcome the bitter aftertaste.

The chemical structures of RA, ST and RD are depicted below.

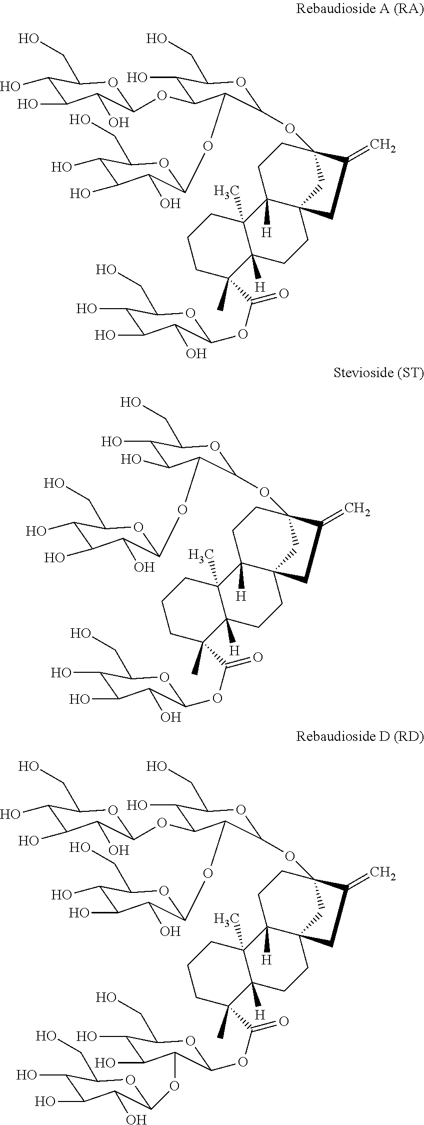

One method of preparing RD is by extraction from Stevia Rebaudian, followed by separation and purification. Because of the extremely low composition (<0.5%) of RD in Stevia Rebaudian, this method can only produce a limited quantity of RD and cannot meet the soaring demand for RD. Another method relates to the semi-synthesis of RD and is disclosed in US2010/0316782 (WO2010/146463). As shown in Scheme 1, RA is converted to Rebaudioside B (RB) by saponification, followed by acetylation to afford acetyl RD. Condensation of acetylated RB with acetylated Sophorose bromide 1 in the presence of silver carbonate provides fully acetylated RD. After de-acetylation using NaOMe, RD is obtained in 8.1% yield.

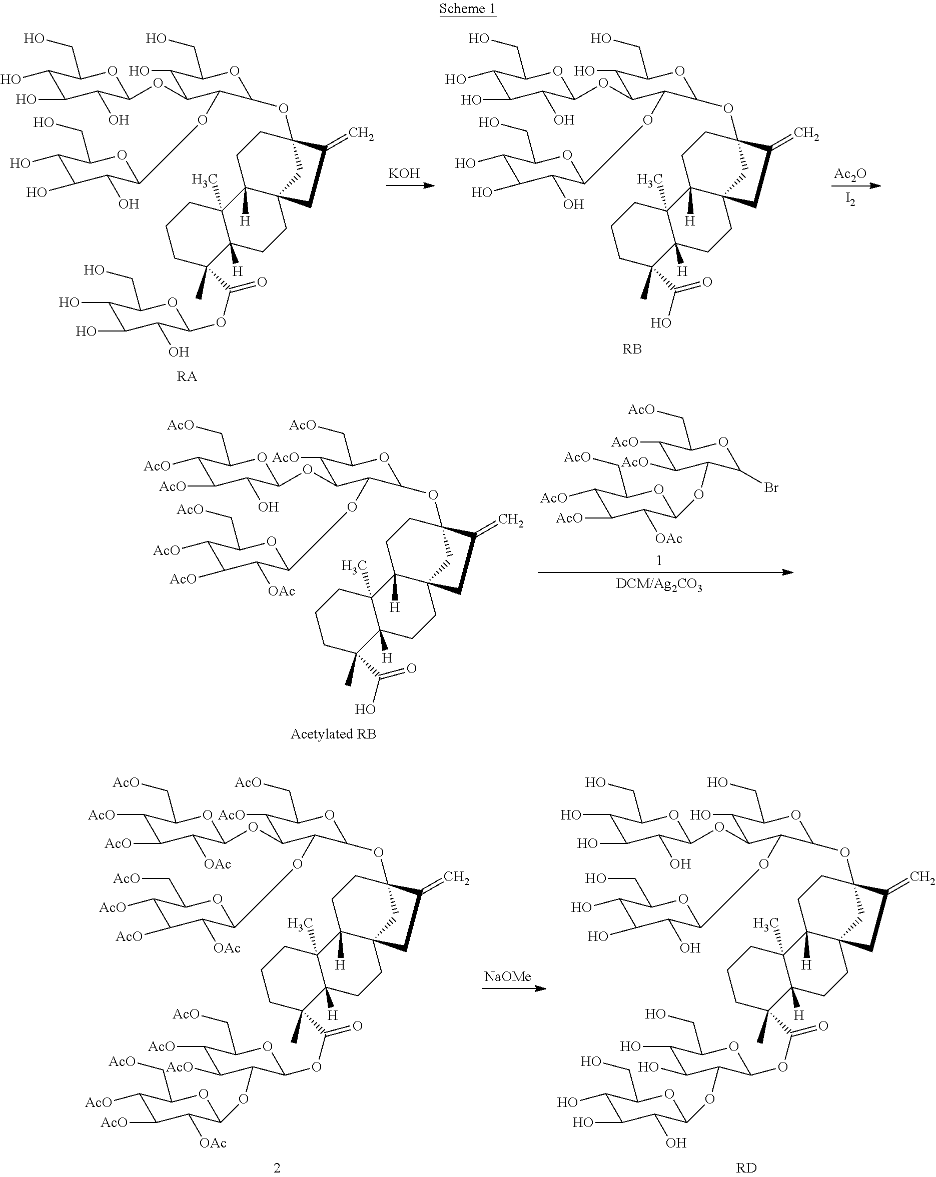

The major drawbacks of this process include, but are not limited to, the following:
1. The yield of reacting acetylated RB with acetylated sophorose bromide 1 by esterification and followed by de-acetylation with NaOMe to afford RD, is as low as 8.1%, which is not suitable for commercial manufacturing.
2. Silver carbonate is required in a key esterification reaction, which results in a high material cost for the process.
3. Introduction of a heavy metal (e.g., silver) in the process increases the risk of unacceptable high level of residual silver metal for human consumption.
4. The purification of final product RD is carried out by column chromatography, which is not suitable for commercial production.

There is a need for an efficient process for making RD and other naturally occurring sweeteners which does not require the use of heavy metal and which is suitable for commercial manufacturing. The present invention relates to an improved process for the preparation of RD and other naturally occurring sweeteners using a novel synthetic strategy, which result in high yields while eliminating the use of heavy metal as a reagent. This process is free of chromatographic purification, and suitable for commercial manufacturing.

### SUMMARY OF THE INVENTION

In one aspect, the present invention provides to a process for preparing a compound of formula (IV) (i.e., RD):

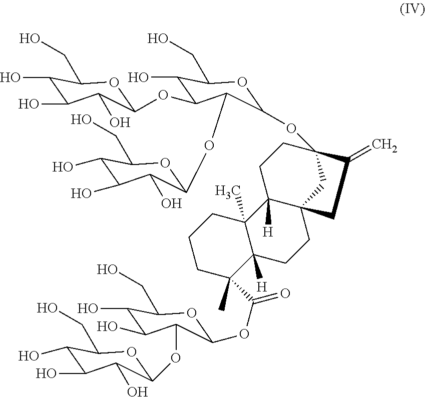

comprising:

(a) selectively hydrolyzing a compound of formula (VII),

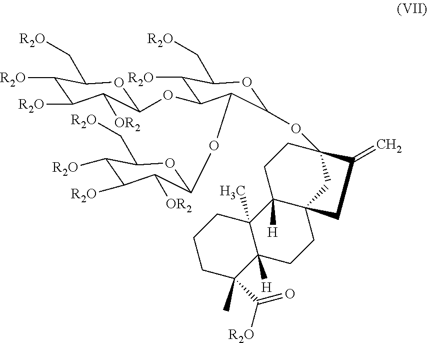

wherein each R₂ is independently a protecting group selected from benzyl, p-methoxybenzyl (PMB), benzyloxy methyl, p-methoxybenzyloxy methyl, (C₁-C₄)alkyl-acyl (e.g. acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

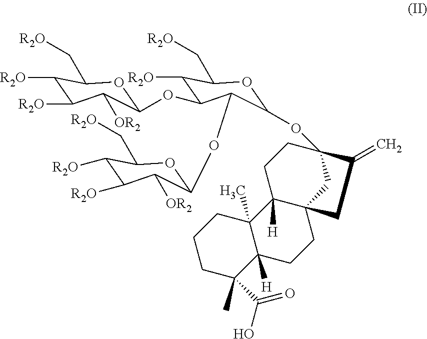

wherein each R₂ is defined as hereinabove;

(b) reacting the compound of formula (II) with a compound of formula (I),

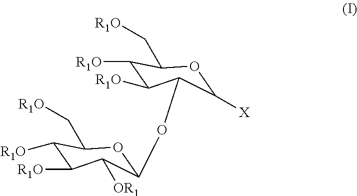

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (III),

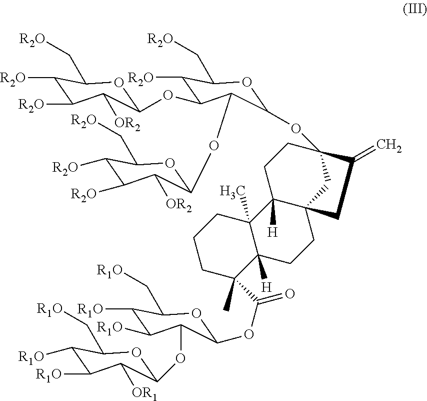

wherein R₁ and R₂ are defined as hereinabove;

(c) removing the protecting groups R₁ and R₂ of the compound of formula (III) to provide the compound of formula (IV).

In another aspect, the present invention provides a process of preparing a compound of formula (X):

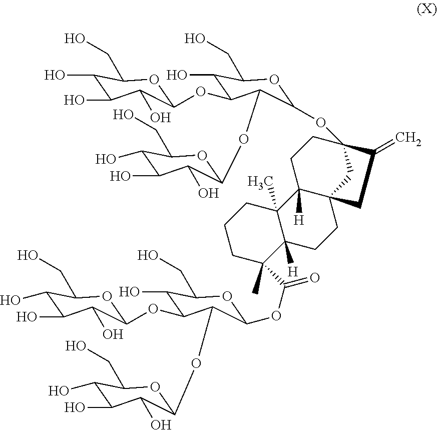

comprising:

(a) selectively hydrolyzing a compound of formula (VII),

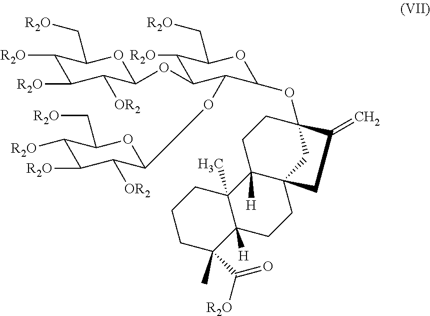

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

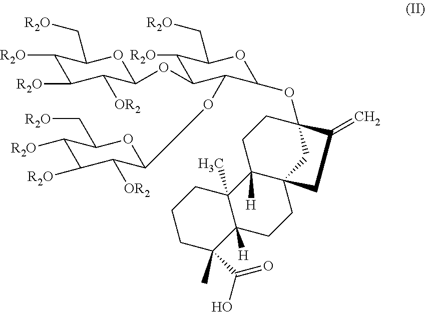

wherein each R₂ is defined as hereinabove;

(b) reacting the compound of formula (II) with a compound of formula (XI),

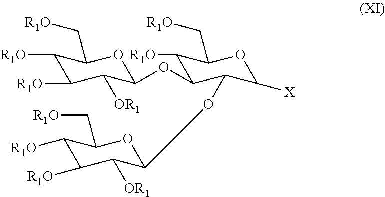

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XII),

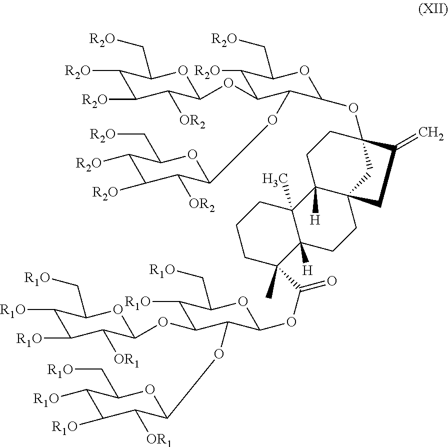

wherein R₁ and R₂ are defined as hereinabove;

(c) removing the protecting groups R₁ and R₂ of the compound of formula (III) to provide the compound of formula (X).

In another aspect, the present invention provides a process for preparing a compound of formula (XIII):

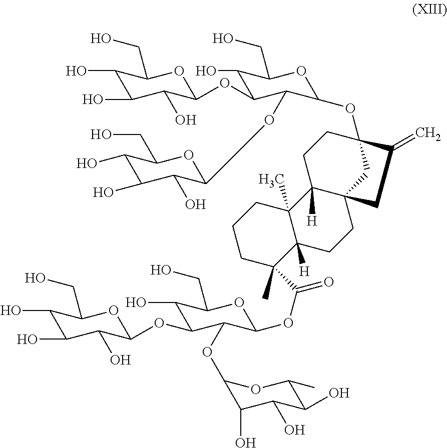

comprising:

(a) selectively hydrolyzing a compound of formula (VII),

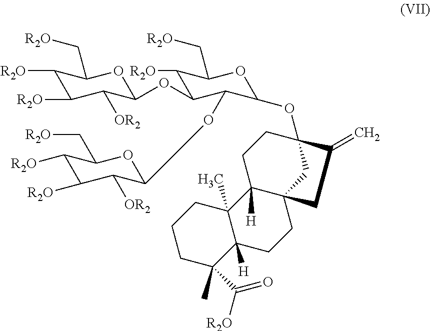

wherein each R₂ is independently a protecting group selected from benzyl, p-methoxybenzyl (PMB), benzyloxy methyl, p-methoxybenzyloxy methyl, (C₁-C₄)alkyl-acyl (e.g. acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

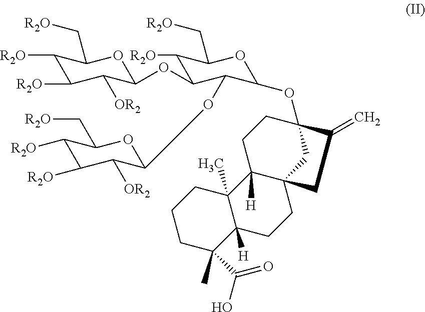

wherein each R₂ is defined as hereinabove;

(b) reacting the compound of formula (II) with a compound of formula (XIV),

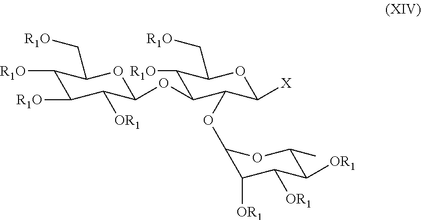

wherein each R₁ is independently a protecting group selected from benzyl, p-methoxybenzyl (PMB), benzyloxy methyl, p-methoxybenzyloxy methyl, (C₁-C₄)alkyl-acyl (e.g. acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XV),

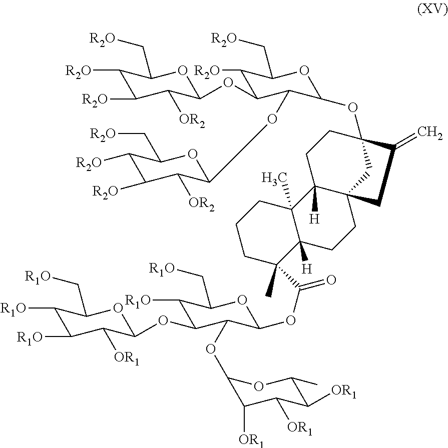

wherein R₁ and R₂ are defined as hereinabove;

(c) removing the protecting groups R₁ and R₂ of the compound of formula (III) to provide the compound of formula (XIII).

In another aspect, the present invention provides a process for preparing a compound of formula (XVI):

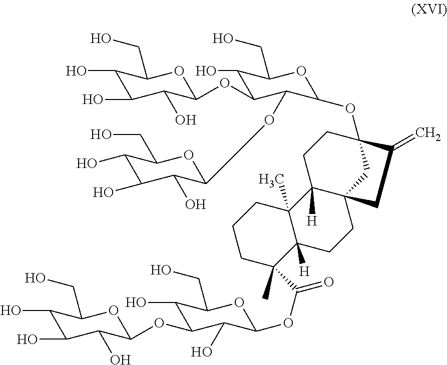

comprising:

(a) selectively hydrolyzing a compound of formula (VII),

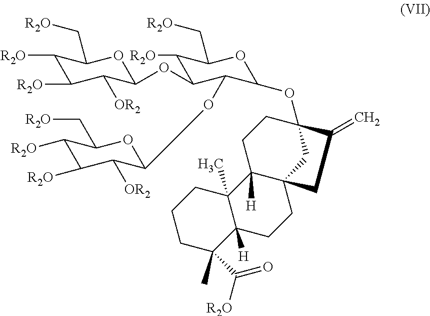

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

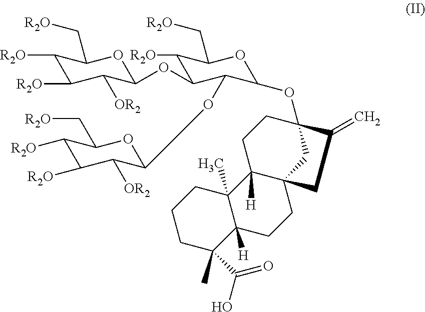

wherein each R₂ is defined as hereinabove;

(b) reacting the compound of formula (II) with a compound of formula (XVII),

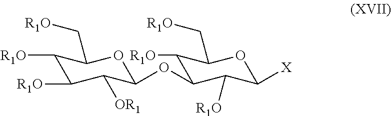

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XVIII),

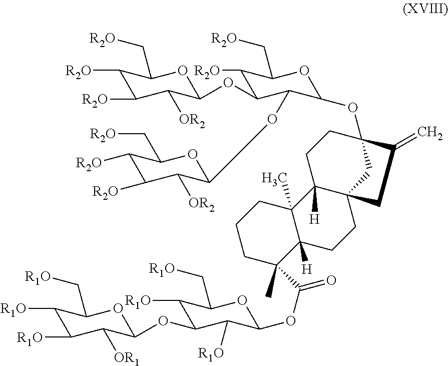

wherein R₁ and R₂ are defined as hereinabove;

(c) removing the protecting groups R₁ and R₂ of the compound of formula (XVIII) to provide the compound of formula (XVI).

In another aspect, the present invention provides a process for preparing a compound of formula (XIX):

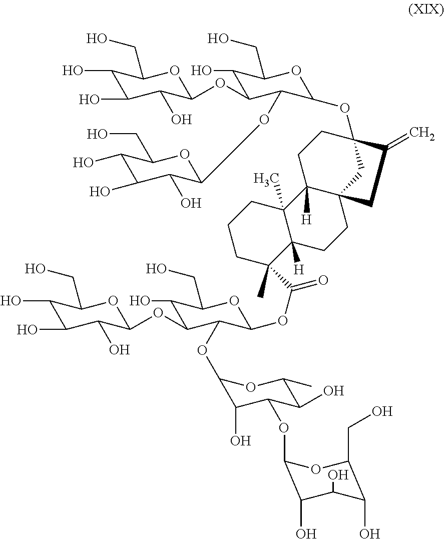

comprising:

(a) selectively hydrolyzing a compound of formula (VII),

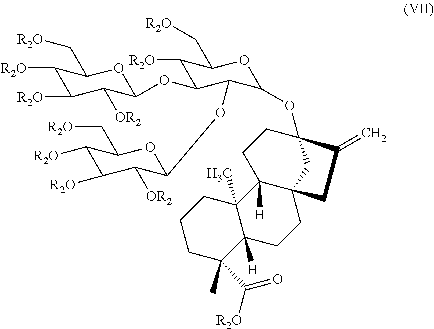

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

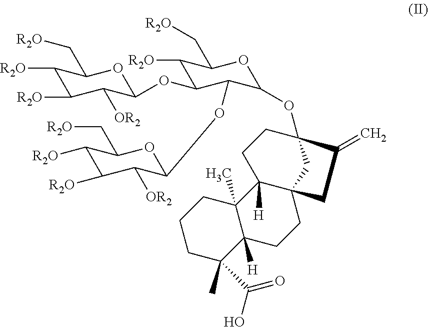

wherein each R₂ is defined as hereinabove;

(b) reacting the compound of formula (II) with a compound of formula (XX),

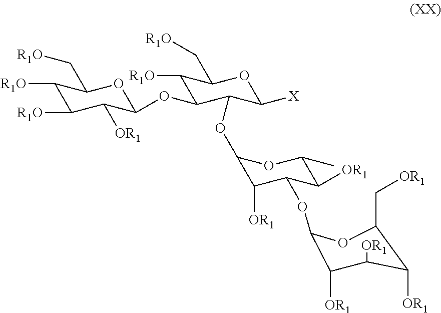

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XXI),

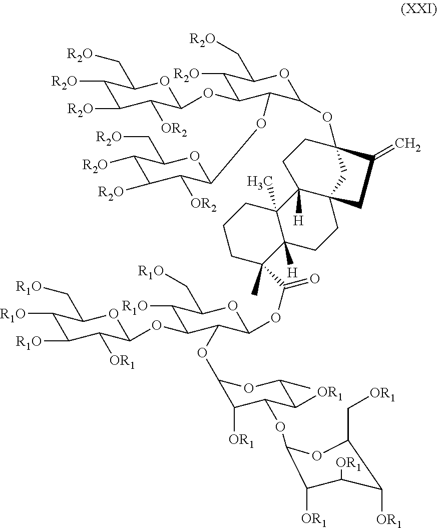

wherein R₁ and R₂ are defined as hereinabove;

(c) removing the protecting groups R₁ and R₂ of the compound of formula (III) to provide the compound of formula (XIX).

In another aspect, the present invention provides a process of preparing a compound of formula (III),

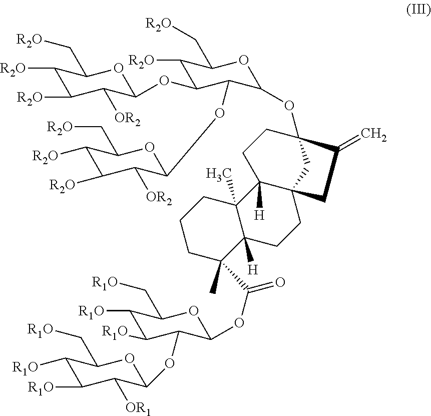

comprising reacting a compound of formula (II),

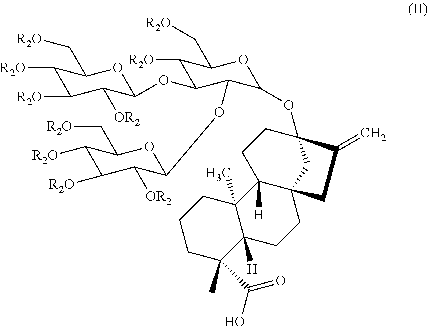

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;

with a compound of formula (Ia),

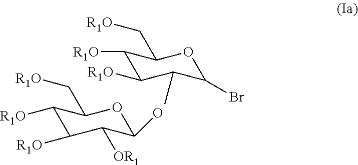

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl; and the anomeric carbon attached with Br can be either α or β configuration; to provide a compound of formula (III) without the use of Ag₂CO₃.

In yet another aspect, the present invention provides a process for a process of preparing a compound of formula (III),

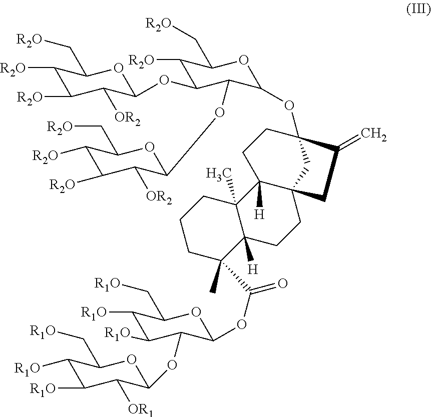

comprising reacting a compound of formula (II),

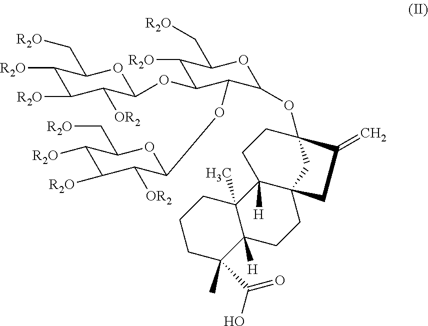

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;

with a compound of formula (Ib),

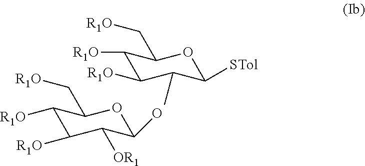

wherein each R₁ is independently a protecting group from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl, and the anomeric carbon attached with S can be either α or β configuration;

to provide a compound of formula (II),

In yet another aspect, the present invention provides a process of preparing a compound of formula (III),

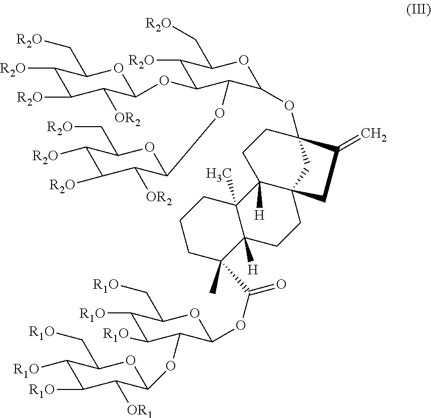

comprising reacting a compound of formula (II),

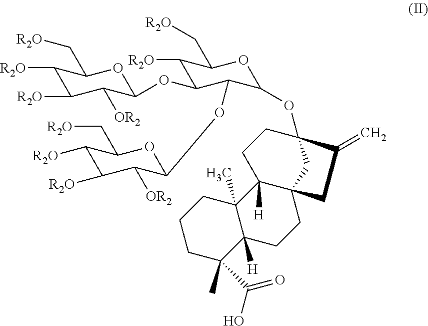

wherein each R₂ independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;

with a compound of formula (Id),

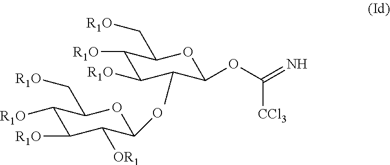

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl; and the anomeric carbon attached with OH can be either α or β configuration;

to provide the compound of formula (III).

In one aspect, the present invention provides to a process for preparing a compound of formula (X) (i.e., Rebaudioside M, RM):

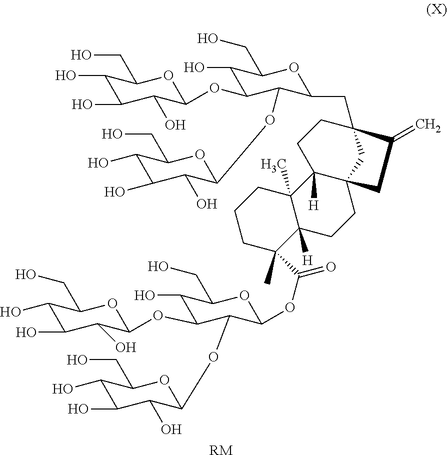

comprising:

(a) reacting the compound of formula (II),

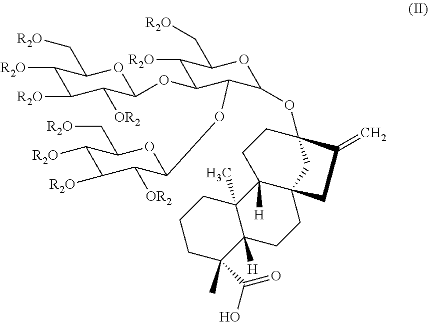

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XI),

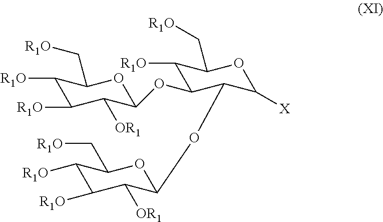

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XII),

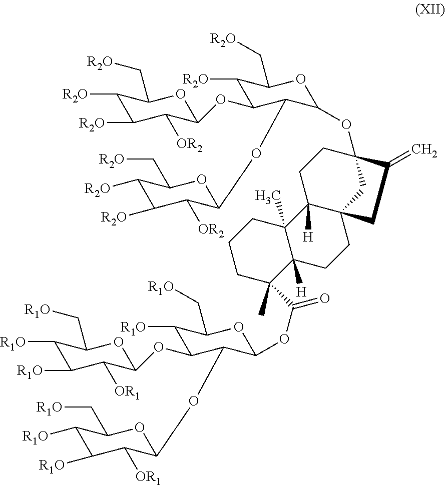

wherein R₁ and R₂ are defined as hereinabove; and

(b) removing the protecting groups R₁ and R₂ of the compound of formula (XII) to provide the compound of formula (X).

In another aspect, the present invention provides to a process for preparing a compound of formula (XIII) (i.e., Rebaudioside N, RN):

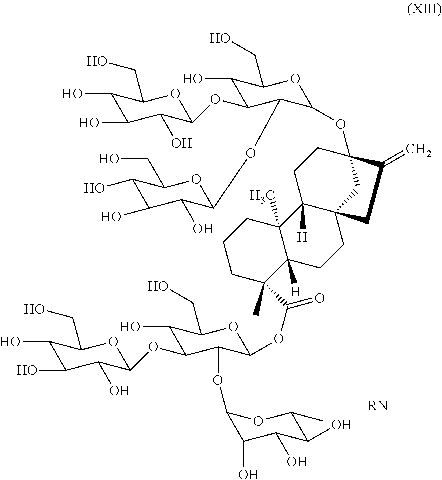

comprising:

(a) reacting the compound of formula (II),

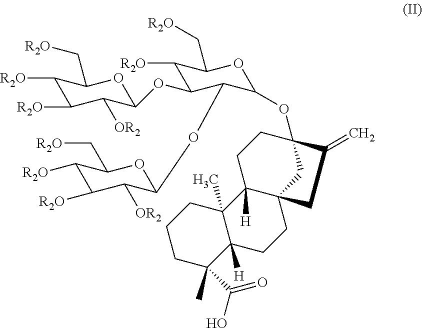

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XIV),

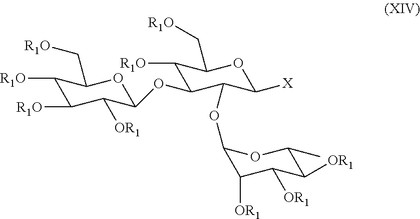

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XV),

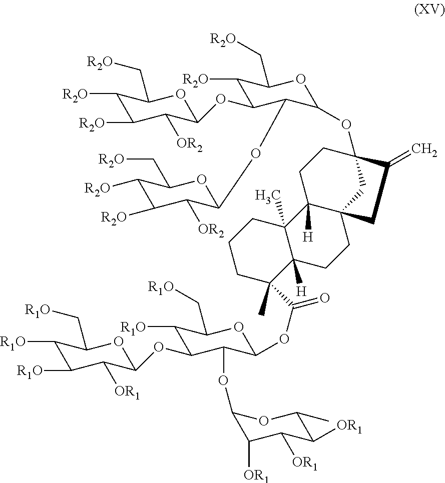

wherein R₁ and R₂ are defined as hereinabove; and

(b) removing the protecting groups R₁ and R₂ of the compound of formula (XV) to provide the compound of formula (XIII).

In another aspect, the present invention provides to a process for preparing a compound of formula (XVI) (i.e., Rebaudioside I, RI):

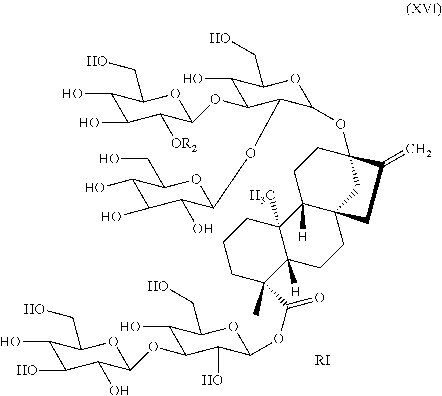

comprising:

(a) reacting the compound of formula (II),

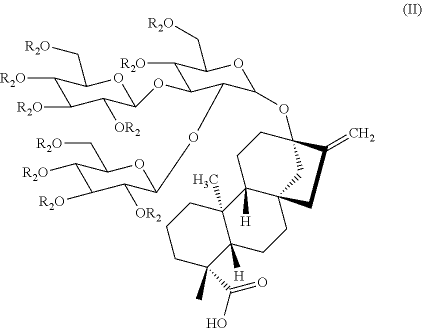

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XVII),

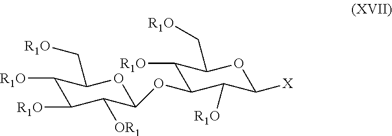

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XVIII),

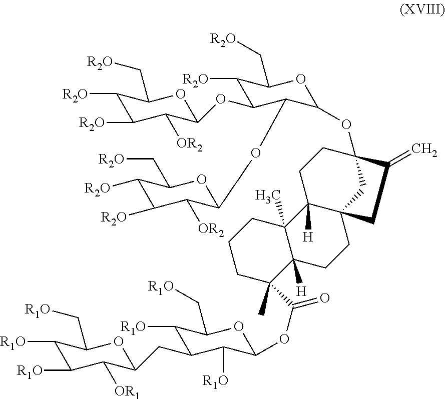

wherein R₁ and R₂ are defined as hereinabove; and

(b) removing the protecting groups R₁ and R₂ of the compound of formula (XVIII) to provide the compound of formula (XVI).

In another aspect, the present invention provides to a process for preparing a compound of formula (XIX) (i.e., Rebaudioside O, RO):

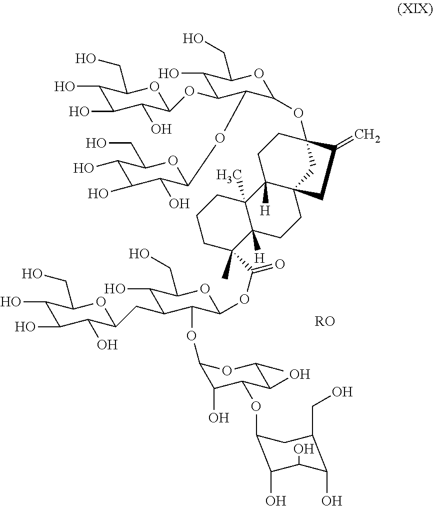

comprising:

(a) reacting the compound of formula (II),

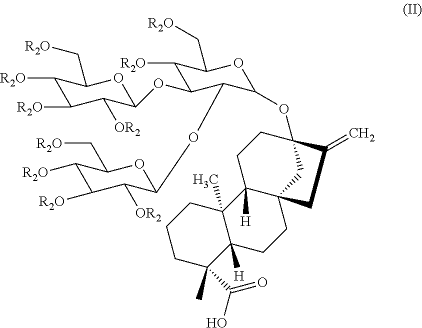

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XX),

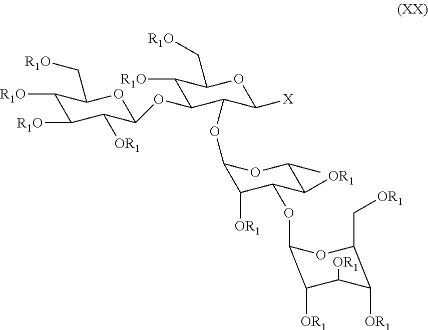

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XXI),

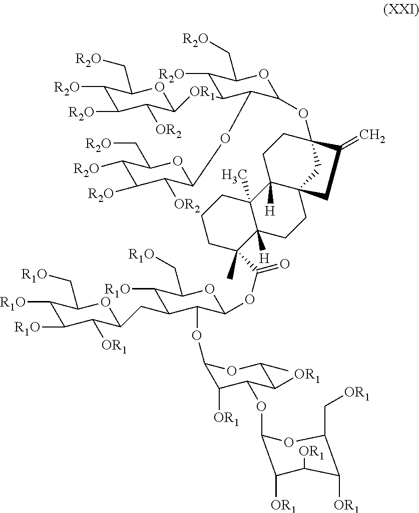

wherein R₁ and R₂ are defined as hereinabove; and

(b) removing the protecting groups R₁ and R₂ of the compound of formula (XXI) to provide the compound of formula (XIX).

### BRIEF DESCRIPTION OF THE DRAWINGS

FIG. 1 depicts ¹H-NMR for compound Ia′.

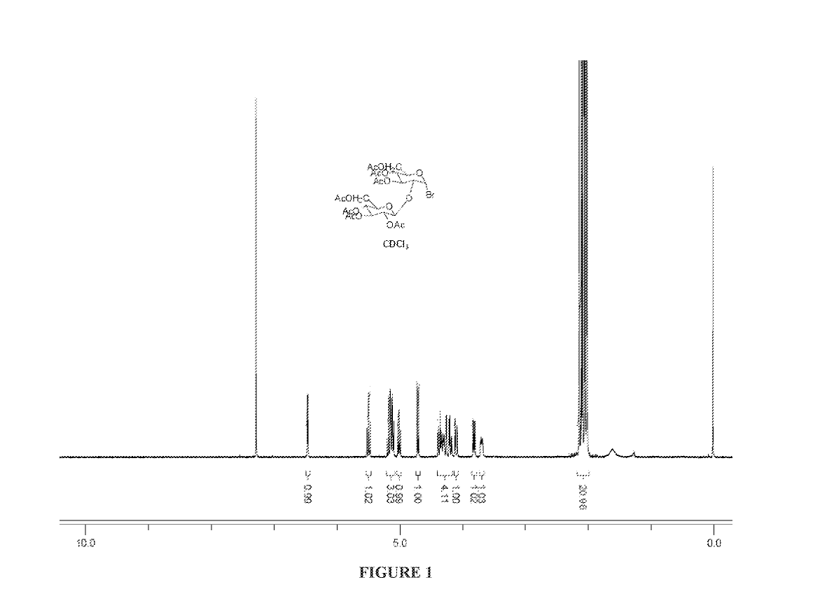

FIG. 2 depicts ¹H-NMR for compound acetylated RB.

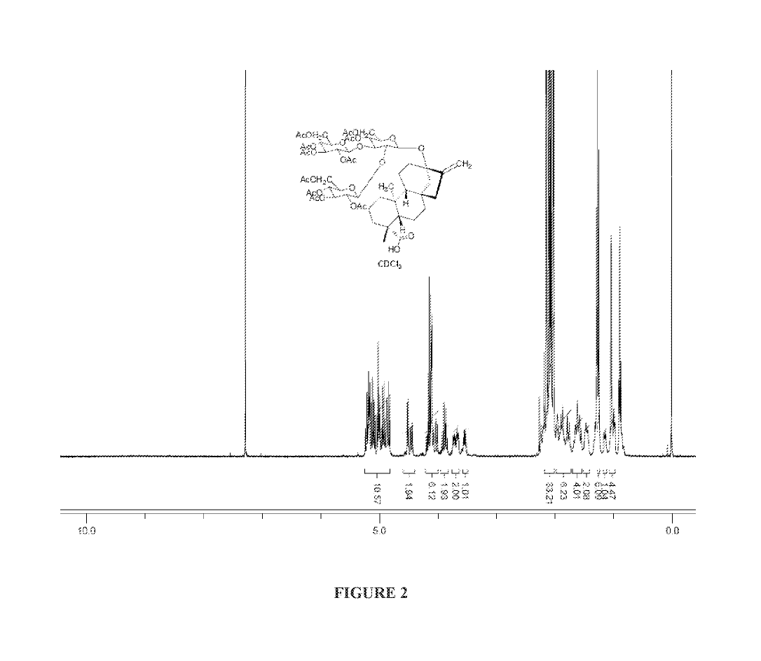

FIG. 3 depicts ¹H-NMR for compound acetylated RD.

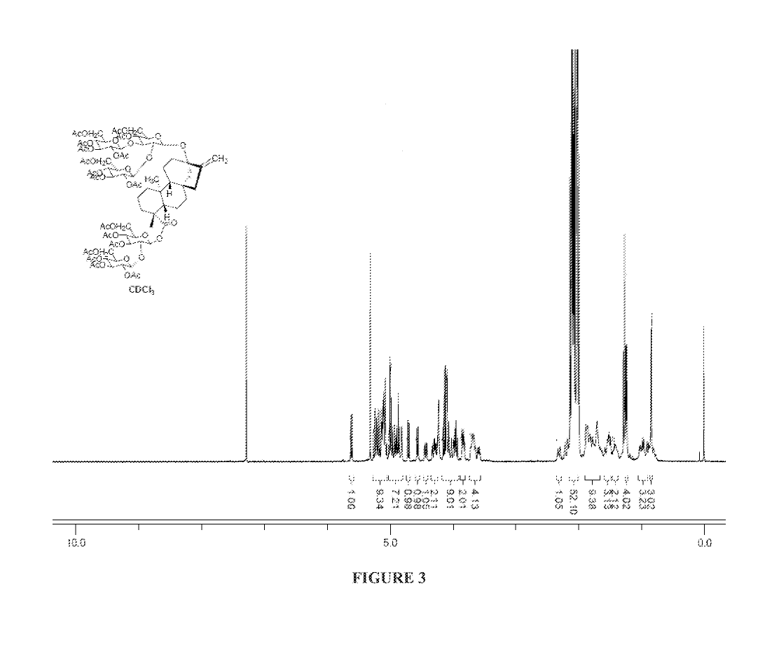

FIG. 4 depicts ¹H-NMR for compound RD.

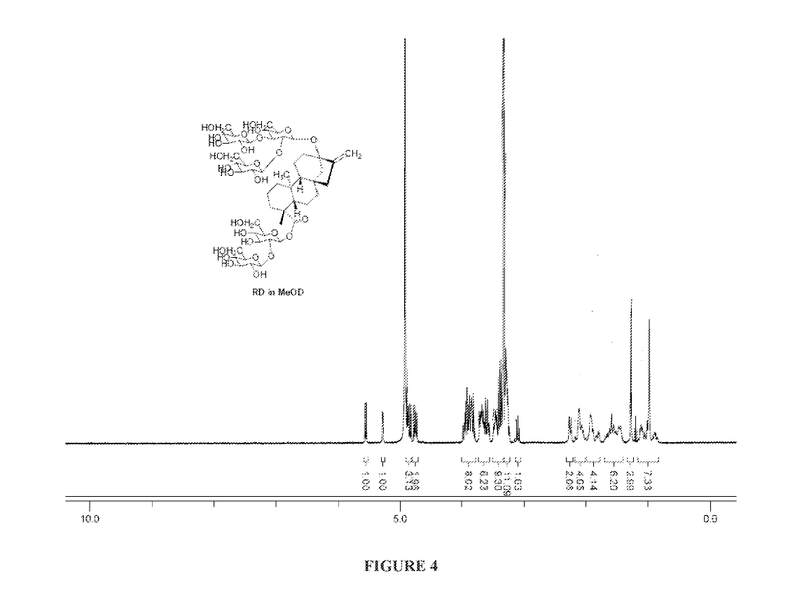

FIG. 5 depicts ¹H-NMR for compound Xia.

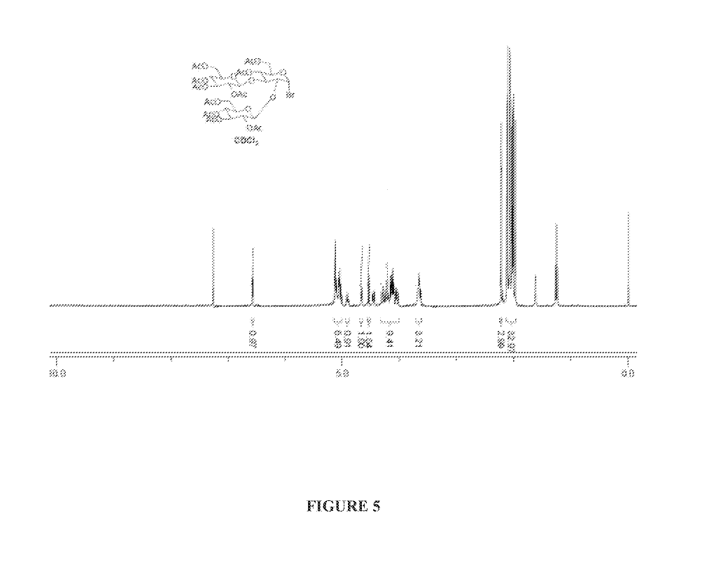

FIG. 6 depicts ¹H-NMR for compound RM.

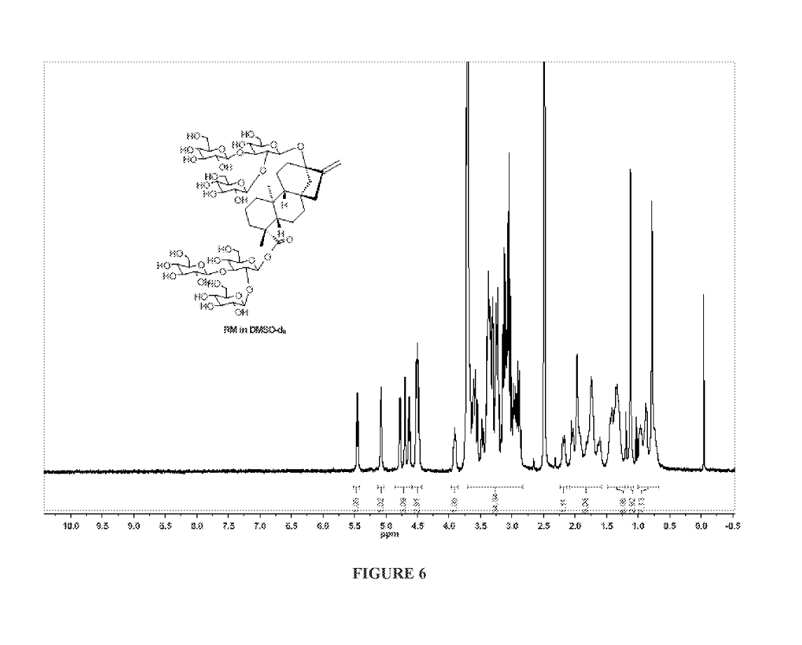

### DETAILED DESCRIPTION OF THE INVENTION

In one aspect, the present invention provides a process for preparing a compound of formula (IV):

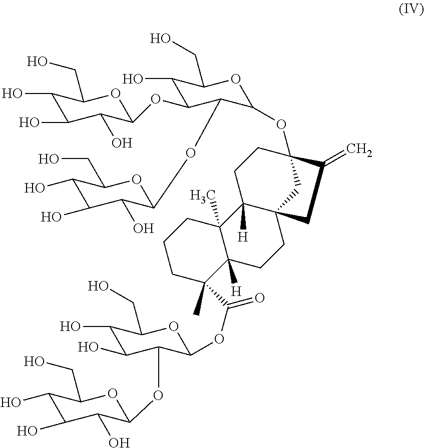

comprising:

(a) selectively hydrolyzing a compound of formula (VII),

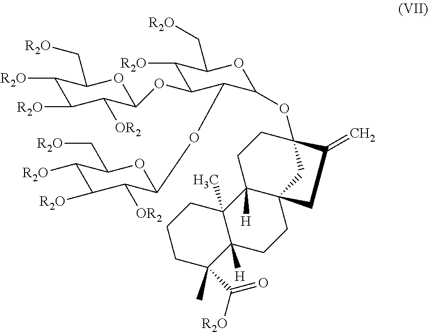

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
to provide a compound of formula (II),

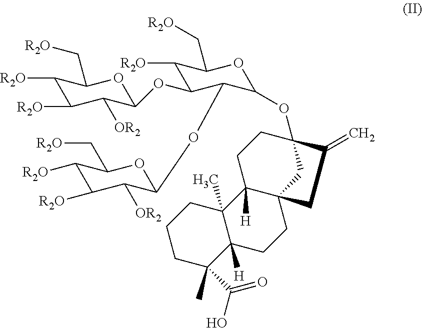

wherein each R₂ is defined as hereinabove;

(b) reacting the compound of formula (II) with a compound of formula (I),

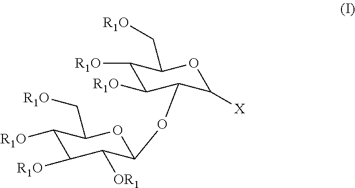

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (III).

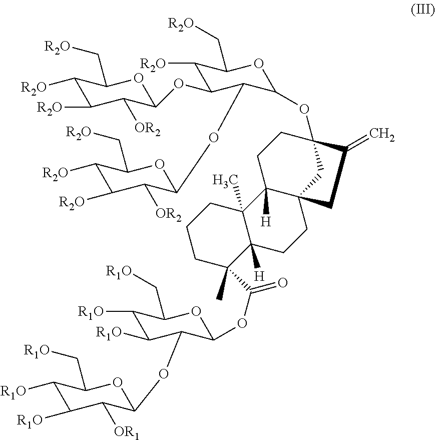

wherein R₁ and R₂ are defined as hereinabove;

(c) removing the protecting groups R₁ and R₂ of the compound of formula (III) to provide the compound of formula (IV).

In another aspect, the present invention provides a process of preparing a compound of formula (X):

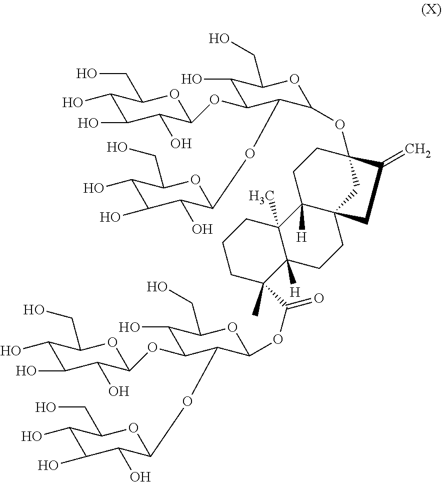

comprising:

(a) selectively hydrolyzing a compound of formula (VII),

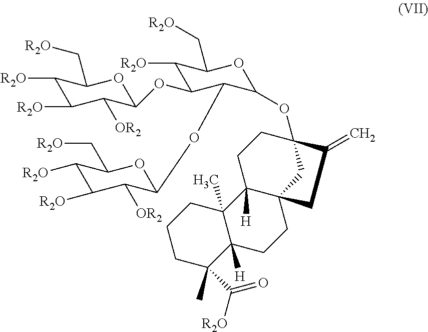

wherein each R₂ is independently a protecting group selected from benzyl, p-methoxybenzyl (PMB), benzyloxy methyl, p-methoxybenzyloxy methyl, (C₁-C₄)alkyl-acyl (e.g. acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

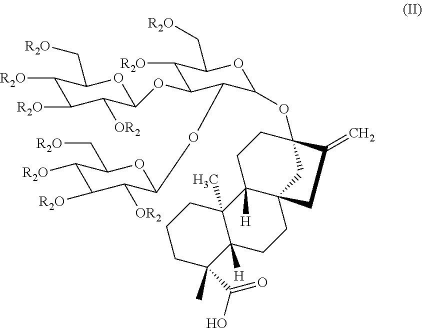

wherein each R₂ is defined as hereinabove;

(b) reacting the compound of formula (II) with a compound of formula (XI),

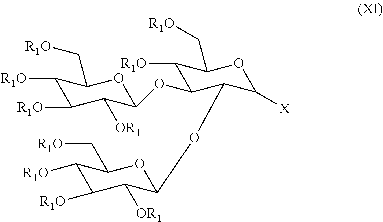

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XII),

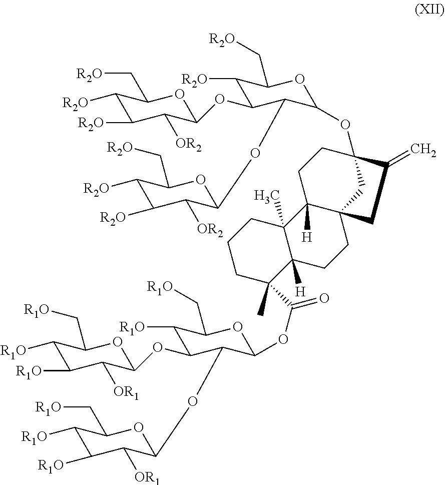

wherein R₁ and R₂ are defined as hereinabove;

(c) removing the protecting groups R₁ and R₂ of the compound of formula (III) to provide the compound of formula (X).

In another aspect, the present invention provides a process for preparing a compound of formula (XIII):

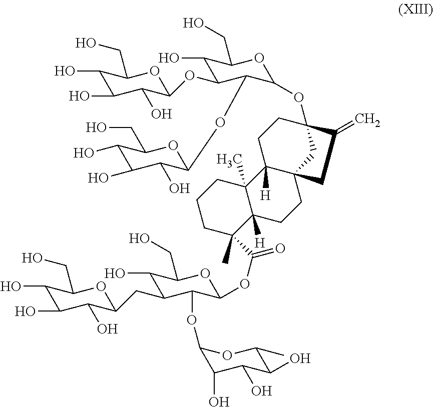

comprising:

(a) selectively hydrolyzing a compound of formula (VII),

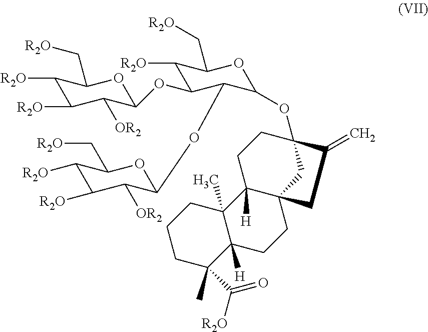

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

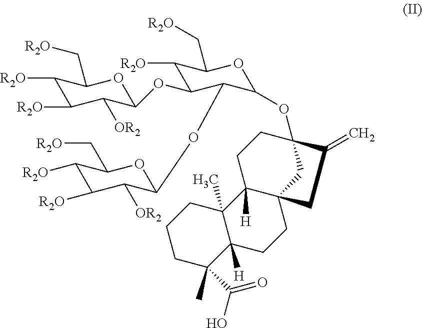

wherein each R₂ is defined as hereinabove;

(b) reacting the compound of formula (II) with a compound of formula (XIV),

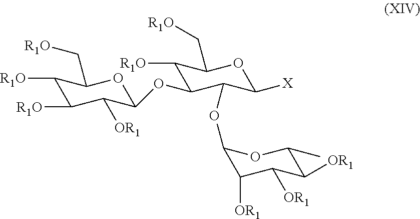

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XV),

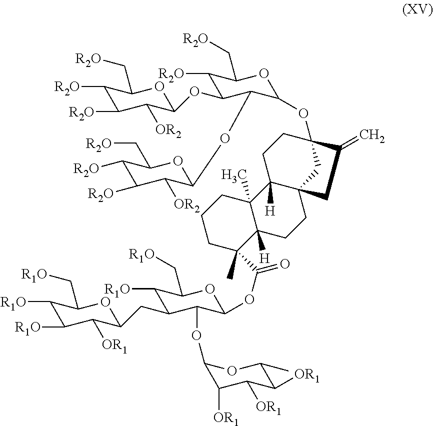

wherein R₁ and R₂ are defined as hereinabove;

(c) removing the protecting groups R₁ and R₂ of the compound of formula (III) to provide the compound of formula (XIII).

In another aspect, the present invention provides a process for preparing a compound of formula (XVI):

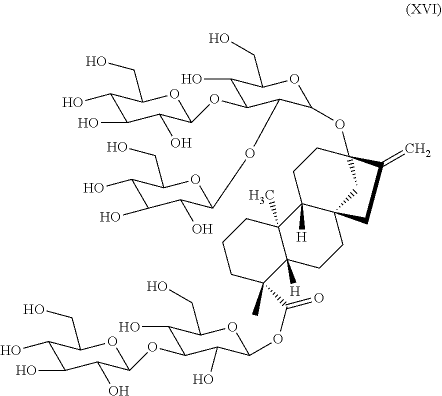

comprising:

(a) selectively hydrolyzing a compound of formula (VII),

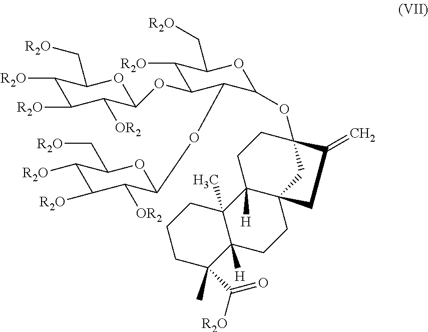

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

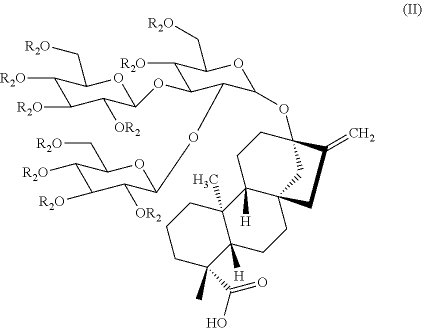

wherein each R₂ is defined as hereinabove;

(b) reacting the compound of formula (II) with a compound of formula (XVII),

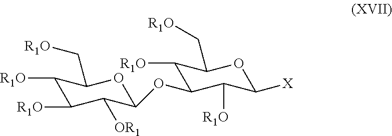

wherein each R₁ is independently a protecting group selected from benzyl, p-methoxybenzyl (PMB), benzyloxy methyl, p-methoxybenzyloxy methyl, (C)—C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂═CH₂, wherein R′ is (C₁-C₄)alkyl or aryl, and the anomeric carbon attached with X can be either α or β configuration;
to provide a compound of formula (XVIII),

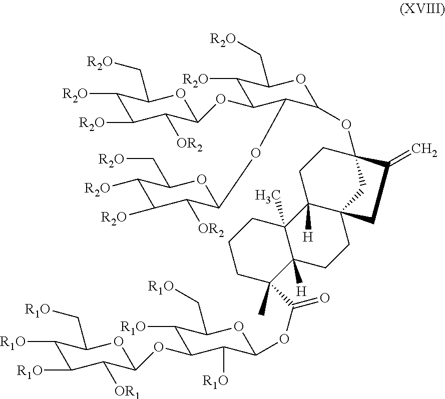

wherein R₁ and R₂ are defined as hereinabove;

(c) removing the protecting groups R₁ and R₂ of the compound of formula (XVIII) to provide the compound of formula (XVI).

In another aspect, the present invention provides a process for preparing a compound of formula (XIX):

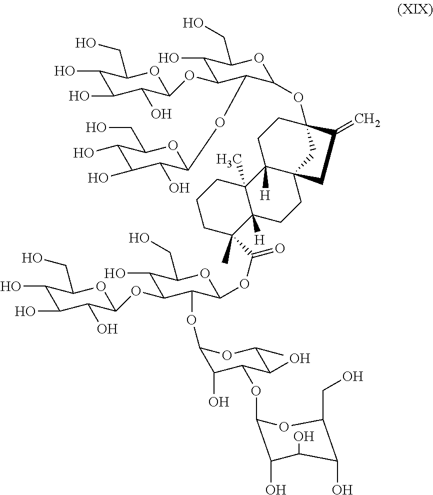

comprising:

(a) selectively hydrolyzing a compound of formula (VII),

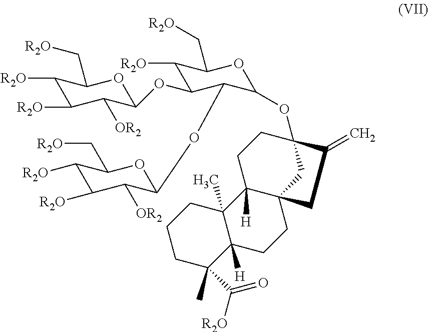

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II),

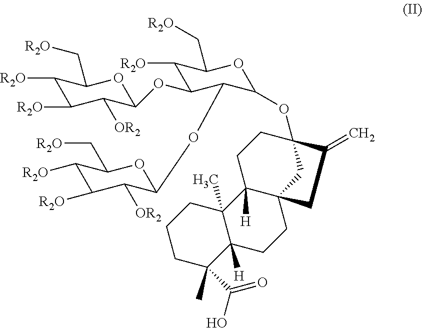

wherein each R₂ is defined as hereinabove;

(b) reacting the compound of formula (II) with a compound of formula (XX),

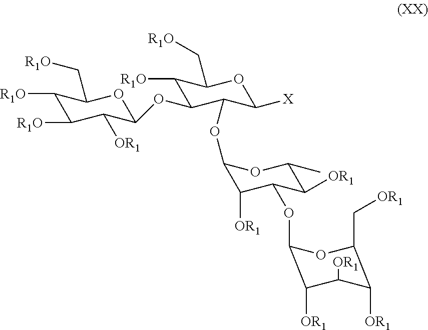

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XXI),

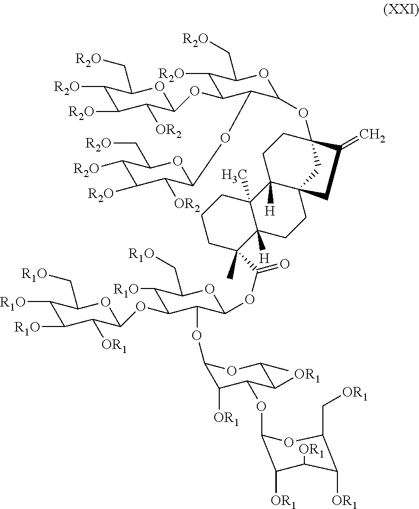

wherein R₁ and R₂ are defined as hereinabove;

(c) removing the protecting groups R₁ and R₂ of the compound of formula (III) to provide the compound of formula (XIX).

In certain embodiments, in step (b), the compound of formula (II) is reacted with the X-substituted intermediate without the use of Ag₂CO₃.

In certain embodiments, each R₁ is independently (C₁-C₄)alkyl-acyl (i.e., (C₁-C₄)alkyl-C(═O)—) or phenyl. In certain other embodiments, each R₂ is independently (C₁-C₄)alkyl-acyl (i.e., (C₁-C₄)alkyl-C(═O)—) or phenyl.

In certain embodiments, each R₁ is independently acetyl (CH₃C(═O)—). In certain other embodiments, each R₂ is independently acetyl (CH₃C(═O)—).

In a further aspect, the present invention provides to a process for preparing a compound of formula (X) (i.e., Rebaudioside M, RM):

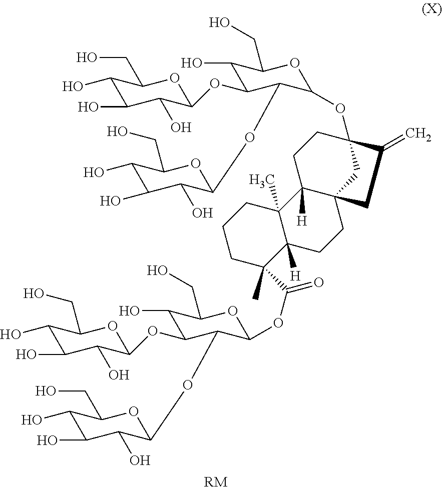

comprising:

(a) reacting the compound of formula (II),

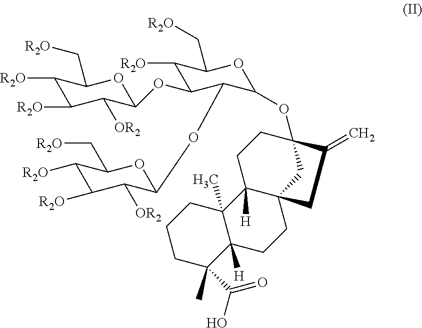

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XI),

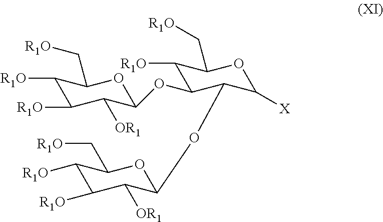

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula XII),

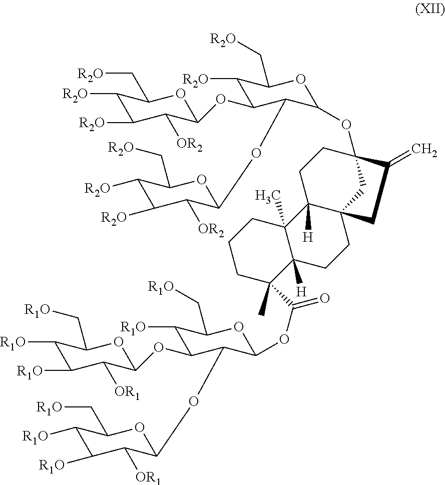

wherein R₁ and R₂ are defined as hereinabove; and

(b) removing the protecting groups R₁ and R₂ of the compound of formula (XII) to provide the compound of formula (X).

In another aspect, the present invention provides to a process for preparing a compound of formula (XIII) (i.e., Rebaudioside N, RN):

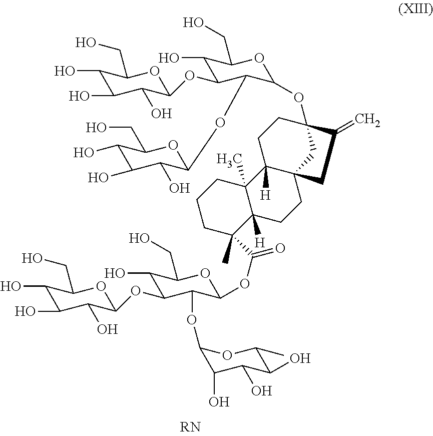

comprising:

(a) reacting the compound of formula (II),

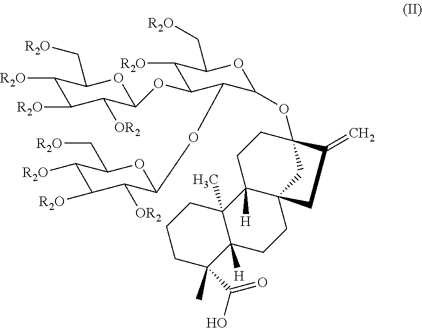

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XIV),

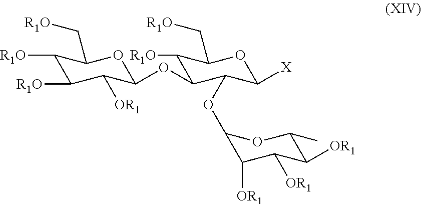

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XV),

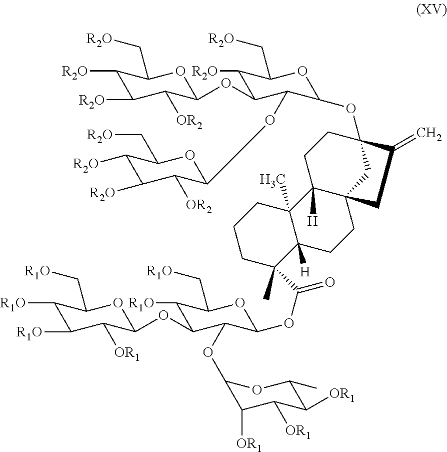

wherein R₁ and R₂ are defined as hereinabove; and

(b) removing the protecting groups R₁ and R₂ of the compound of formula (XV) to provide the compound of formula (XIII).

In another aspect, the present invention provides to a process for preparing a compound of formula (XVI) (i.e., Rebaudioside I, RI):

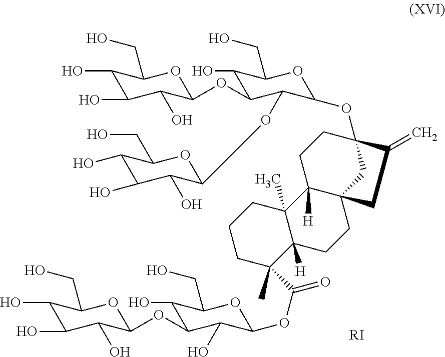

comprising:

(a) reacting the compound of formula (II),

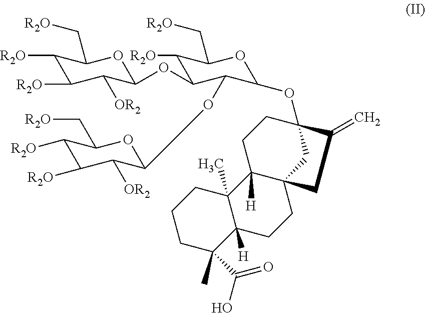

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XVII),

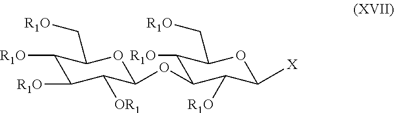

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XVIII),

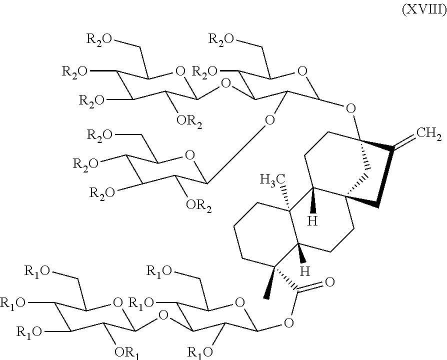

wherein R₁ and R₂ are defined as hereinabove; and

(b) removing the protecting groups R₁ and R₂ of the compound of formula (XVIII) to provide the compound of formula (XVI).

In another aspect, the present invention provides to a process for preparing a compound of formula (XIX) (i.e. Rebaudioside O, RO):

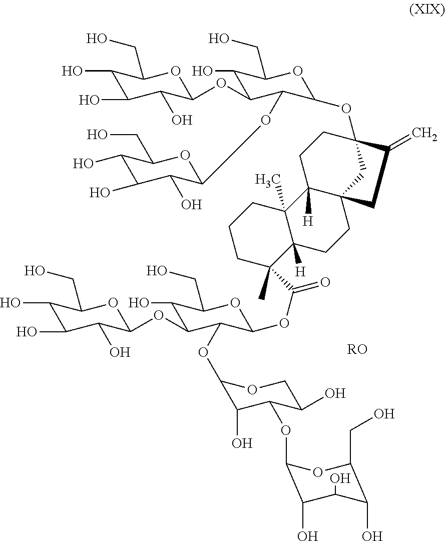

comprising:

(a) reacting the compound of formula (II),

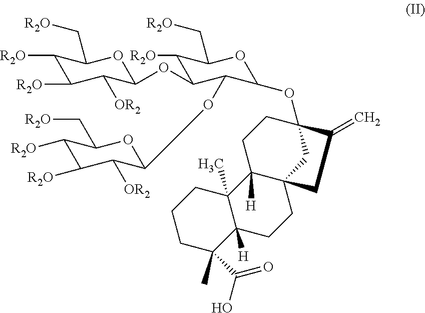

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
with a compound of formula (XX),

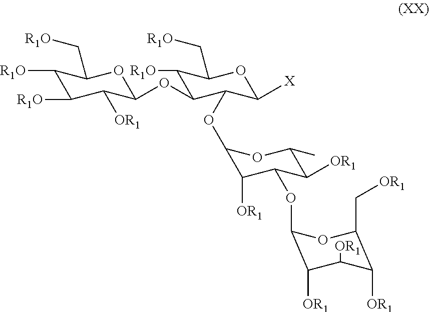

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XXI),

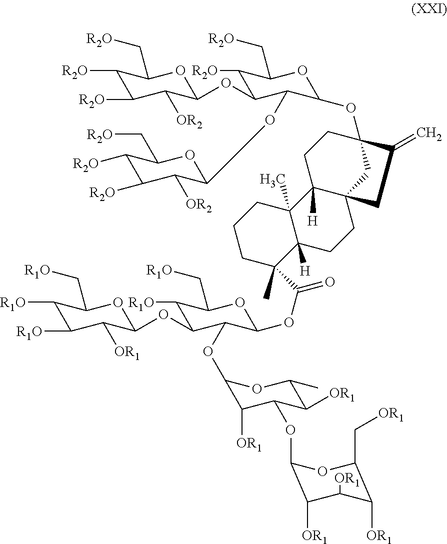

wherein R₁ and R₂ are defined as hereinabove; and

(b) removing the protecting groups R₁ and R₂ of the compound of formula (XXI) to provide the compound of formula (XIX).

In certain embodiments, in step (a), the compound of formula (II) is reacted with the X-substituted intermediate without the use of Ag₂CO₃.

In certain embodiments, each R₁ is independently (C₁-C₄)alkyl-acyl (i.e., (C₁-C₄)alkyl-C(═O)—) or phenyl. In certain other embodiments, each R₂ is independently (C₁-C₄)alkyl-acyl (i.e., (C₁-C₄)alkyl-C(═O)—) or phenyl.

In certain embodiments, each R₁ is independently acetyl (CH₃C(—O)—). In certain other embodiments, each R₂ is independently acetyl (CH₃C(═O)—).

In certain embodiments, the compound of formula (VII) is prepared by:

(a) hydrolyzing a compound of formula (VIII),

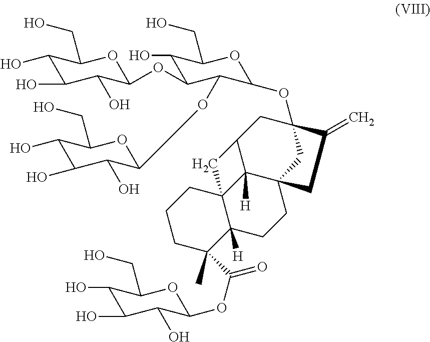

to provide a compound of formula (IX),

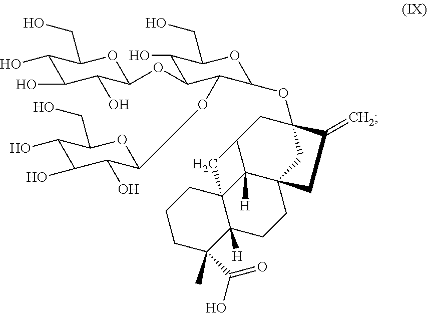

and

(b) protecting the OH groups of the compound of formula (IX) to provide the compound of formula (VII),

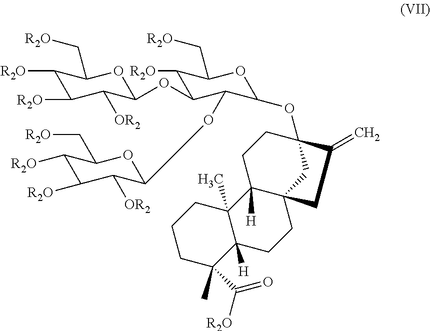

wherein each R₂ is independently a protecting group selected from benzyl, p-methoxybenzyl (PMB), benzyloxy methyl, p-methoxybenzyloxy methyl, (C₁-C₄)alkyl-acyl (e.g. acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl.

In certain embodiments, each R₂ is independently (C₁-C₄)alkyl-acyl (i.e., (C₁-C₄)alkyl-C(—O)—) or phenyl. In certain other embodiments, each R₂ is independently acetyl (CH₃C(═O)—).

In one aspect, the present invention provides a compound of formula (IV) prepared by the processes as described hereinabove.

In another aspect, the present invention provides a process of preparing a compound of formula (III),

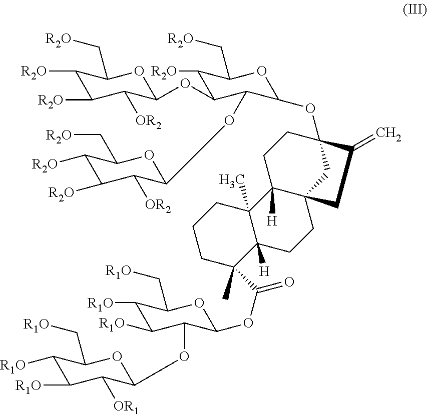

comprising reacting a compound of formula (II),

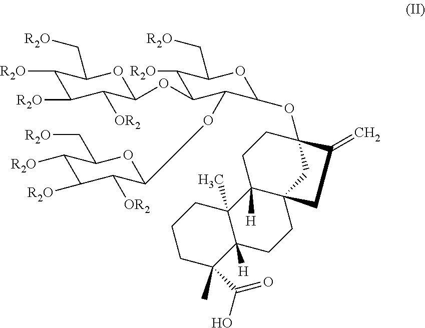

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl; with a compound of formula (Ia),

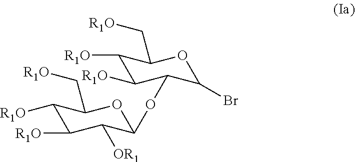

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl; and the anomeric carbon attached with Br can be either α or β configuration;

to provide a compound of formula (III) without the use of Ag₂CO₃.

In certain embodiments, the anomeric carbon attached with Br is α configuration.

In certain embodiments, the compound of formula (II) is reacted with the compound of formula (Ia) in the presence of a phase transferring reagent and an inorganic base.

In certain embodiments, the phase transferring reagent is TBAB, TBAC, TBAI, or TEBAC. In certain other embodiments, the inorganic base is KHCO₃, K₂CO₃, K₃PO₄, or KH₂PO₄.

In one aspect, the present invention provides a compound of formula (III) prepared by the processes as described hereinabove.

In another aspect, the present invention provides a process of preparing a compound of formula (III),

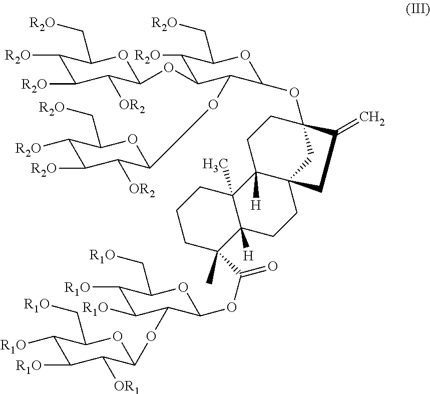

comprising reacting a compound of formula (II),

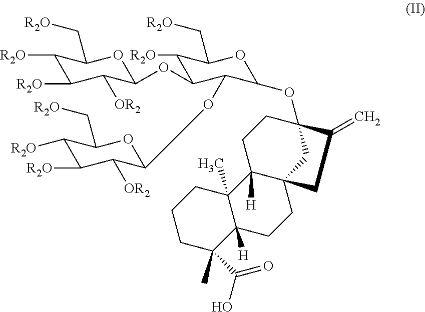

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl; with a compound of formula (Ib),

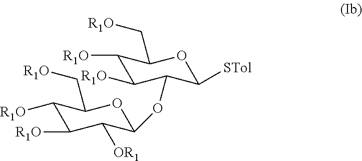

wherein each R₁ is independently a protecting group from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl, and the anomeric carbon attached with S can be either α or β configuration:

to provide a compound of formula (III).

In certain embodiments, the anomeric carbon attached with Br is α configuration.

In certain embodiments, the compound of formula (II) is reacted with the compound of formula (Ib) in the presence of NIS (N-iodosuccinimide) and an acid. In certain embodiments, the acid is a Lewis acid. In certain other embodiments, the acid is a Lewis acid selected from TBSOTf (CF₃SO₃SiMe₂t-Bu). TMSOTf (trifluoromethanesulfonic acid trimethylsilylester), TfOH, BF₃:Et₂O, and IDCP (iodonium dicollidine perchlorate).

In one aspect, the present invention provides a compound of formula (III) prepared by the processes as described hereinabove.

In yet another aspect, the present invention provides a process of preparing a compound of formula (III),

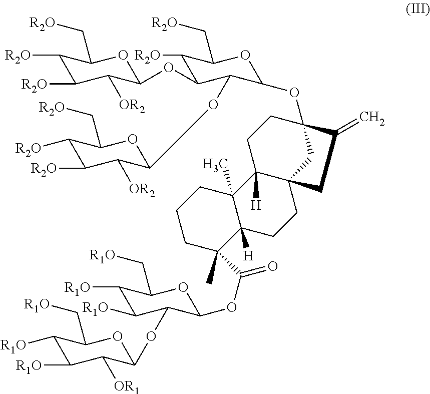

comprising reacting a compound of formula (II),

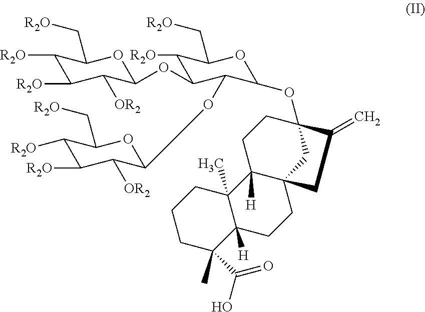

wherein each R₂ independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;

with a compound of formula (Id),

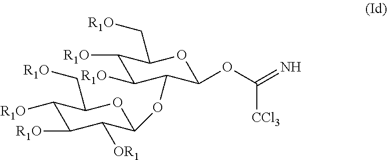

wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl; and the anomeric carbon attached with OH can be either α or β configuration; to provide the compound of formula (III).

In certain embodiments, the anomeric carbon attached with Br is α configuration.

The certain embodiments, the compound of formula (II) is reacted with the compound of formula (Id) in the presence of a Lewis acid. In certain other embodiments, the acid is a Lewis acid selected from TBSOTf (CF₃SO₃SiMe₂t-Bu), TMSOTf (trifluoromethanesulfonic acid trimethylsilylester). TfOH, BF₃:Et₂O, and IDCP (iodonium dicollidine perchlorate). In yet other embodiments, the Lewis acid is TBSOTf (CF₃SO₃SiMe₂t-Bu), TMSOTf, or TfOH.

In one aspect, the present invention provides a compound of formula (III) prepared by the processes as described hereinabove.

The certain embodiments, the protecting groups R₁ and R₂ of the compound of formula (III) as described hereinabove can be removed via, e.g. hydrolysis, to provide a compound of formula (IV),

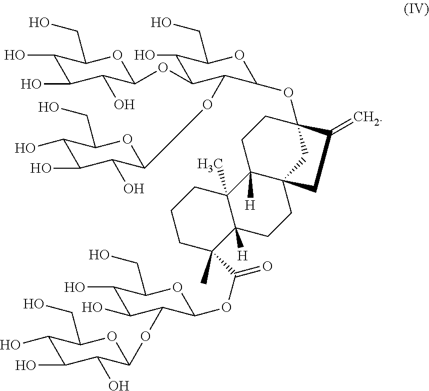

In certain embodiments, the compound of formula (II),

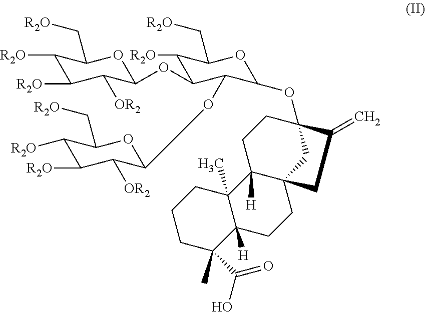

is prepared by selectively hydrolyzing a compound of formula (VII),

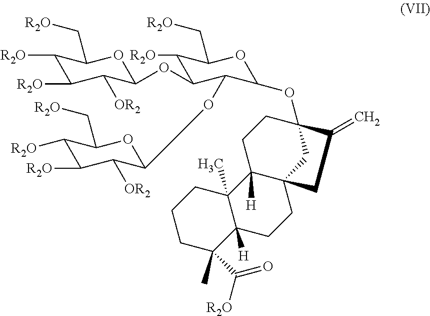

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl.

In certain embodiments, each R₂ is independently (C₁-C₄)alkyl-acyl (i.e., (C₁-C₄)alkyl-C(═O)—) or phenyl. In certain other embodiments, each R₂ is independently acetyl (CH₃C(═O)—).

In certain embodiments, the compound of formula (VII),

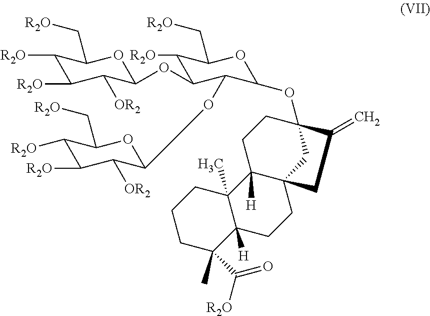

is prepared by:

(a) hydrolyzing a compound of formula (VIII),

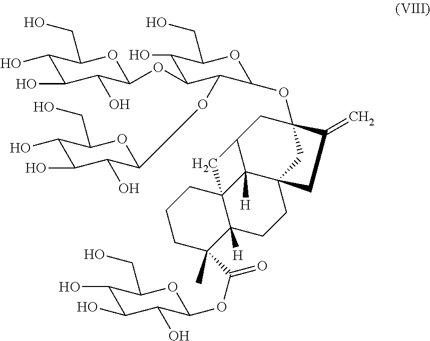

to provide a compound of formula (IX),

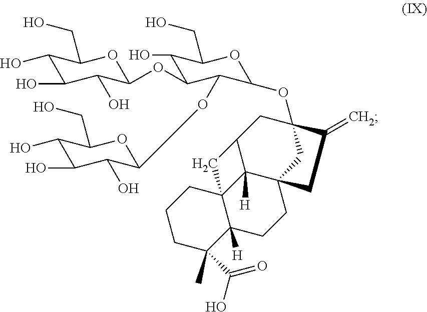

and

(b) protecting the OH groups of the compound of formula (IX) to provide the compound of formula (VII),

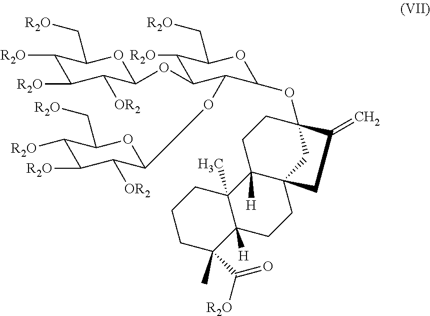

wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl.

In certain embodiments, each R₂ is independently (C₁-C₄)alkyl-acyl (i.e., (C₁-C₄)alkyl-C(—O)—) or phenyl. In certain other embodiments, each R₂ is independently acetyl (CH₃C(═O)—).

In a further aspect, the present invention provides a compound of Examples 1-8 prepared by the processes as described therein.

Methods of Preparation

In the following schemes, the protecting groups R₁ and R₂ are defined as hereinabove. Other suitable OH protecting groups can be found in Greene and Wuts. Protective Groups in Organic Synthesis, 3^{rd} Ed., 1999, which is incorporated by reference in its entirety.

As illustrated in Scheme 2, the compound of Formula (Ib) is prepared by reacting the compound of Formula (Ia) with toluenethiol (TolSH). The compound of Formula (Ic) is prepared by NaI-catalyzed hydrolysis of the compound of Formula (Ia). The compound of formula (Id) is obtained by condensation of the compound of Formula (Ic) with trichloroacetonitrile in the presence of DBU. The compound of Formula (Ia) (wherein R₁=acetyl) is a derivative of sophorose and can be prepared from Stevioside (ST) by a literature procedure (J.A.C.S. 78, 4709, 1957).

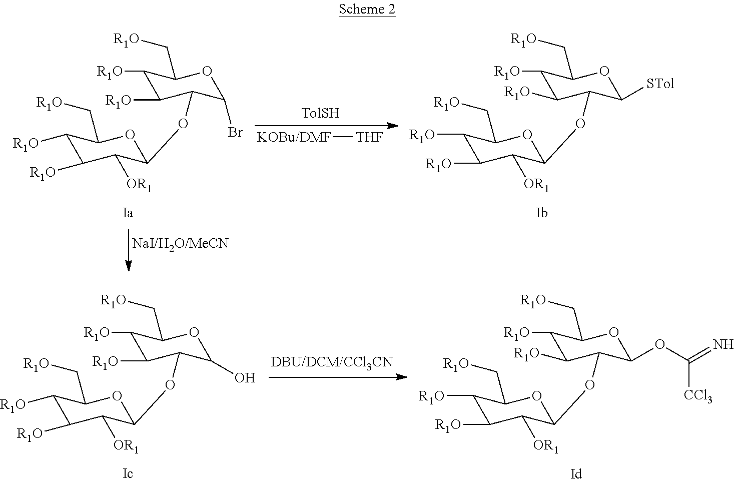

As shown in Scheme 3, the compound of formula (III) can be obtained by reacting the compound of formula (II) with the compound of formula (Ia) in the presence of a phase transfer reagent and an inorganic base. Advantageously, this step does not require the use of Ag₂CO₃, a potentially toxic heavy metal. The phase transferring reagent includes, but is not limited to, TBAB, TBAC, TBAI, or TEBAC; and the inorganic base is KHCO₃, K₂CO₃, K₃PO₄, or KH₂PO₄. The inorganic base includes, but is not limited to, an alkaline salt of carbonate, phosphate or its buffer solution, e.g. KHCO₃, K₂CO₃, K₃PO₄, or KH₂PO₄.

Alternatively, the compound of formula (III) can be obtained by reacting the compound of formula (II) with the compound of formula (Ib) in the presence of NIS and an acid, such as a Lewis acid. The Lewis acid includes, but is not limited to, TBSOTf (CF₃SO₃SiMe₂t-Bu), TMSOTf (trifluoromethanesulfonic acid trimethylsilylester), TfOH, BF₃:Et₂O, and IDCP (iodonium dicollidine perchlorate). Furthermore, the compound of formula (III) can be obtained by reacting the compound of formula (II) with the compound of formula (Id) in the presence of a Lewis acid such as TBSOTf (CF₃SO₃SiMe₂t-Bu), TMSOTf, or TfOH.

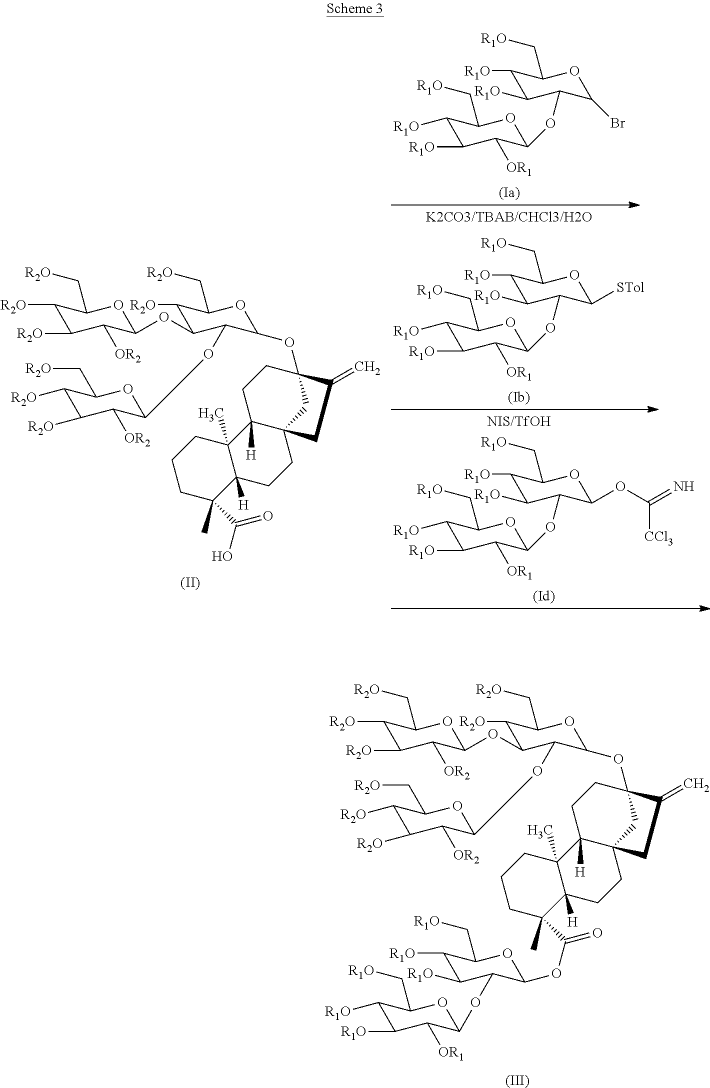

According to Scheme 4, the compound of Formula (II) is obtained by selective hydrolysis of the compound of Formula (VII). In certain embodiments, selective hydrolysis can be achieved by reacting the compound of Formula (VII) with an acid. Non-limiting examples of the acid include HCl, H₃PO₄, NaH₂PO₄, KH₂PO₄, BF₃.Et₂O, and HBF₄.

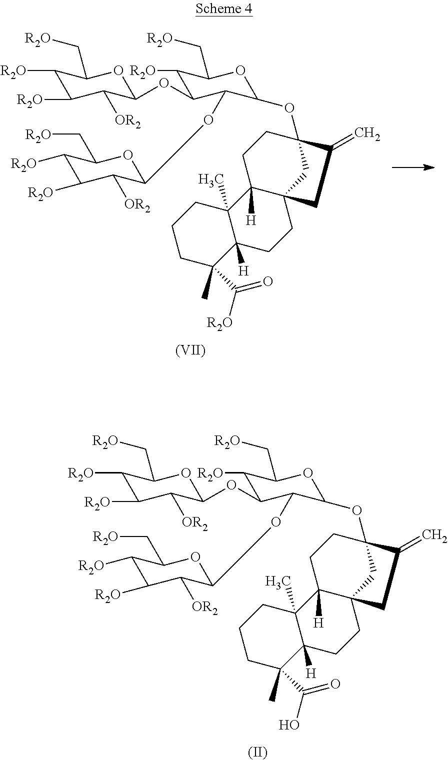

As shown in Scheme 5, the compound of Formula (VII) is prepared by hydrolysis of the compound of Formula (VIII) (i.e., RA) to afford the compound of Formula (IX), followed by protecting the OH groups of the compound of Formula (IX). In certain embodiments, the conversion from the compound of formula (IX) to the compound of formula (VII) and then to the compound of formula (I) (in Scheme 4) can be done in one pot synthesis.

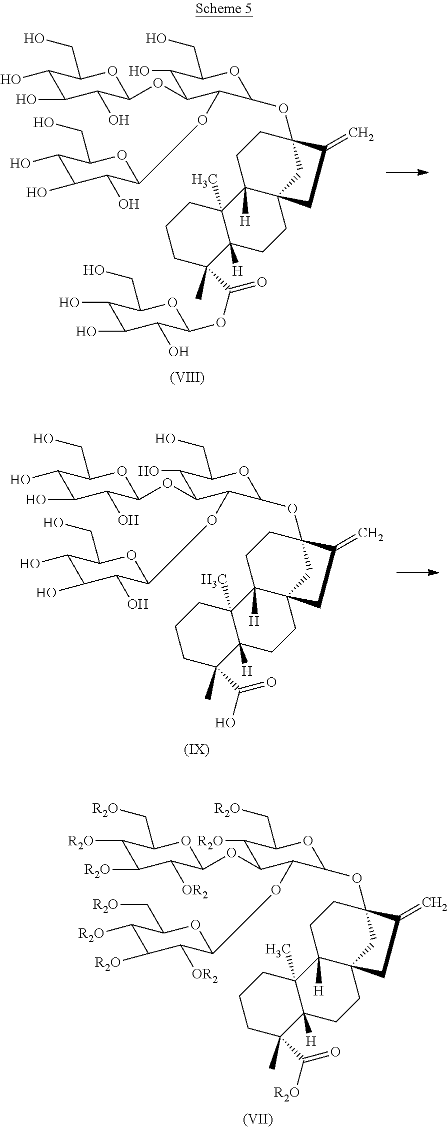

According to Scheme 6, the compound of formula (IV) (i.e., RD) is obtained by removing the protecting groups of R₁ and R₂ of the compound of Formula (III). In certain embodiments, the removal is achieved by hydrolysis. Purification of final product RD can be performed by general purification methods, such as extraction, washing, crystallization, column chromatography and re-crystallization. In certain embodiments, alcohol/water is used for the re-crystallization. In certain other embodiments, ethyl alcohol/water is used for the re-crystallization.

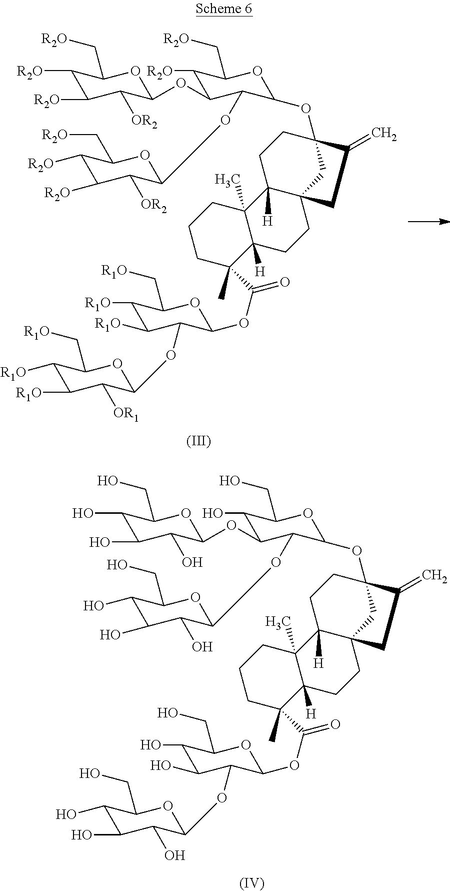

As illustrated in Scheme 7, the compound of formula IX (RA) is protected to give the compound of formula (XXII), which is selectively hydrolyzed to give the compound of formula (XXIII). Treatment of compound of formula (XXIII) with an acid such as hydrogen bromide affords the compound of formula (XI) which further reacts with the compound of formula (II) to yield the compound of formula (XII), followed by subsequent hydrolysis to provide the compound of formula (X) (RM).

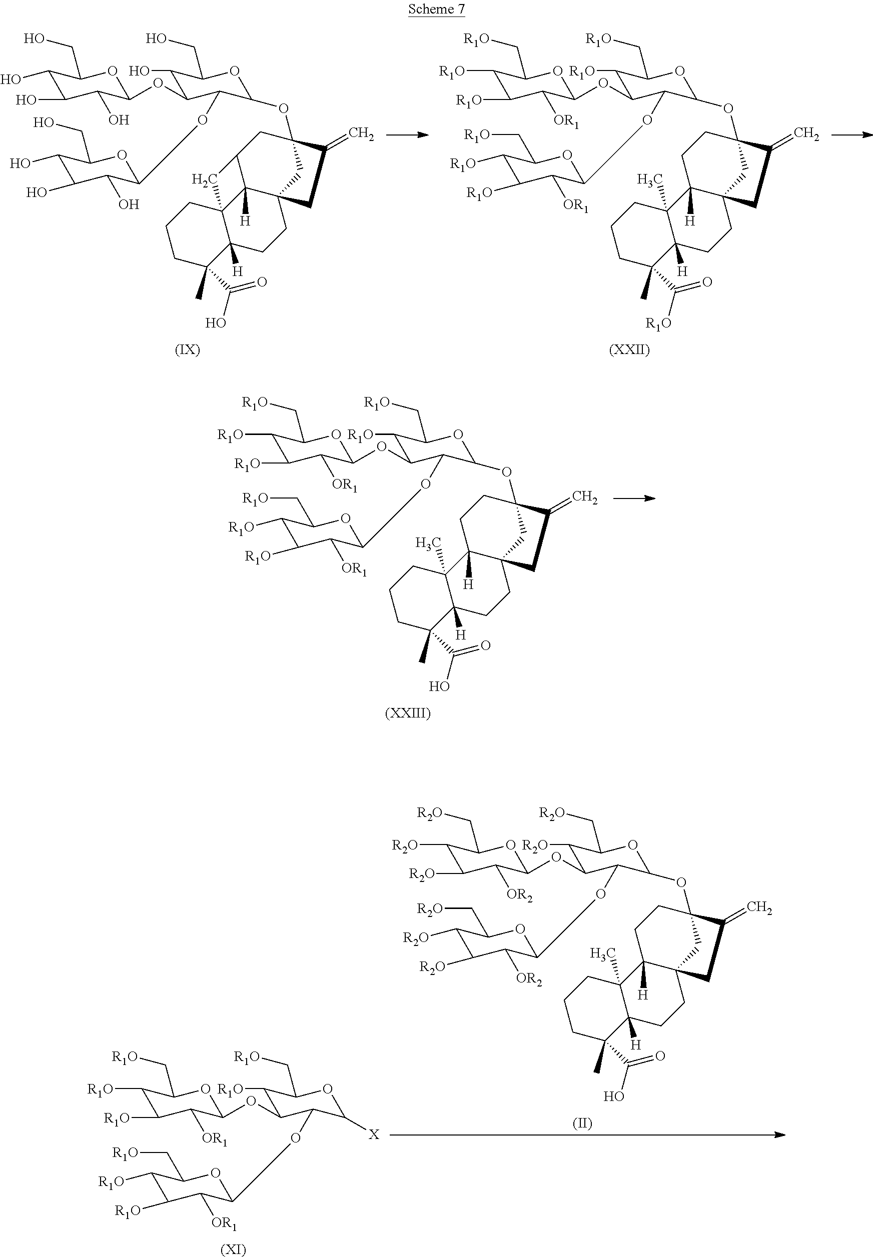

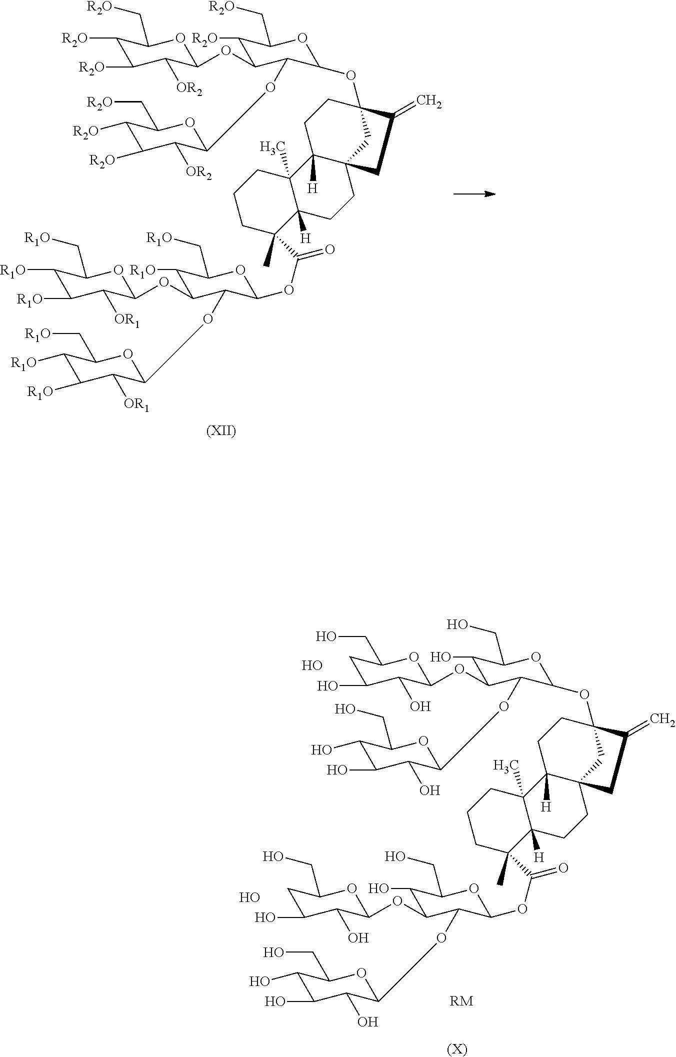

The synthesis of the compound of formula (XIII) (RN) is shown in Scheme 8. Hydrolysis of the compound of formula (XXIV) (Rebaudioside C, RC) gives the compound of formula (XXV) which is protected and followed by selective hydrolysis to yield the compound of formula (XXVI). Conversion of the compound of formula (XXVI) by treatment of an acid (such as HBr) affords the compound of formula (XIV) which further reacts with the compound of formula (II) to provide the compound of formula (XV). Deprotection of the compound of formula (XV) yields the compound of formula (XIII) (RN).

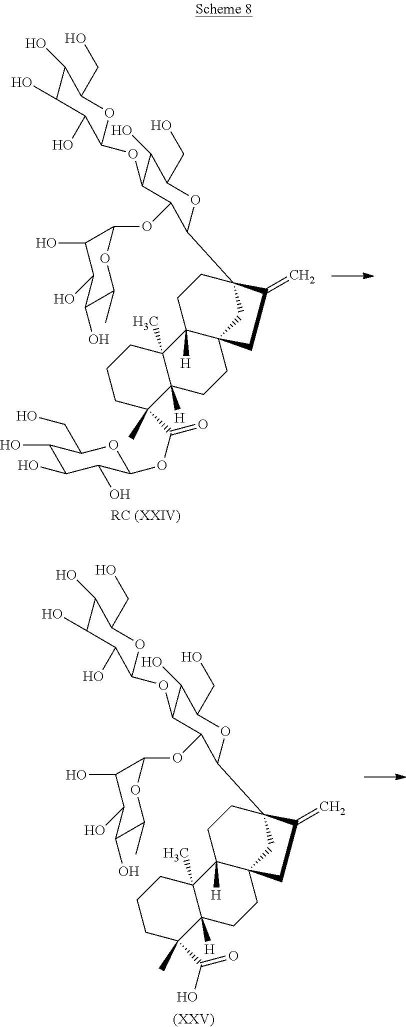

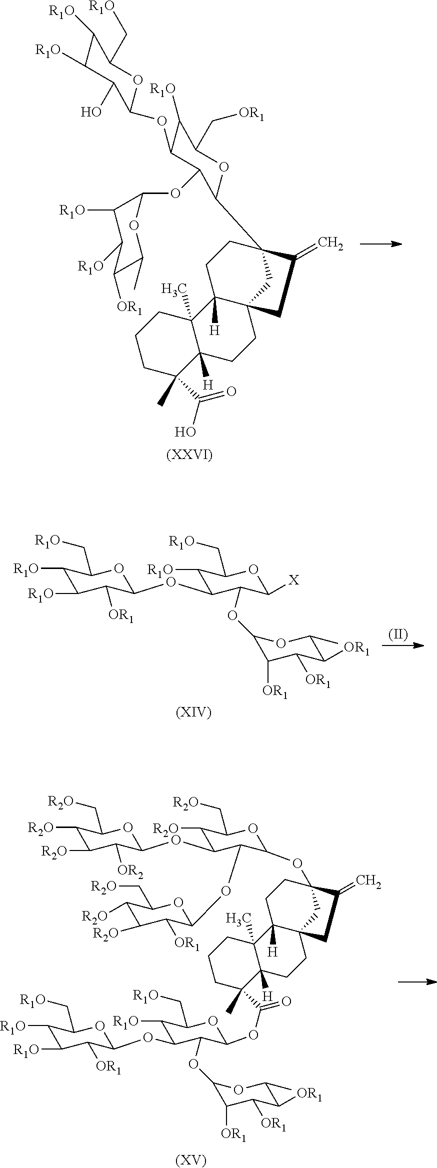

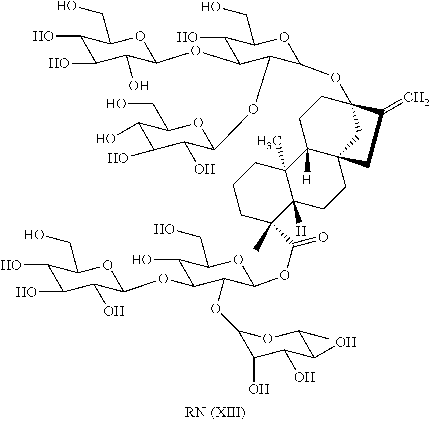

The syntheses of compound RI and RO are summarized in Schemes 9 and 10 using a method analogous to that for the preparation of RD. The compound of formula (II) reacts with the compounds of formulae (XVII) and (XX) respectively to give the corresponding compounds of formulae (XVIII) and (XXI). Deprotection of the compounds of formulae (XVIII) and (XXI) yields desired natural sweeteners RI (XVI) and RO (XIX), respectively.

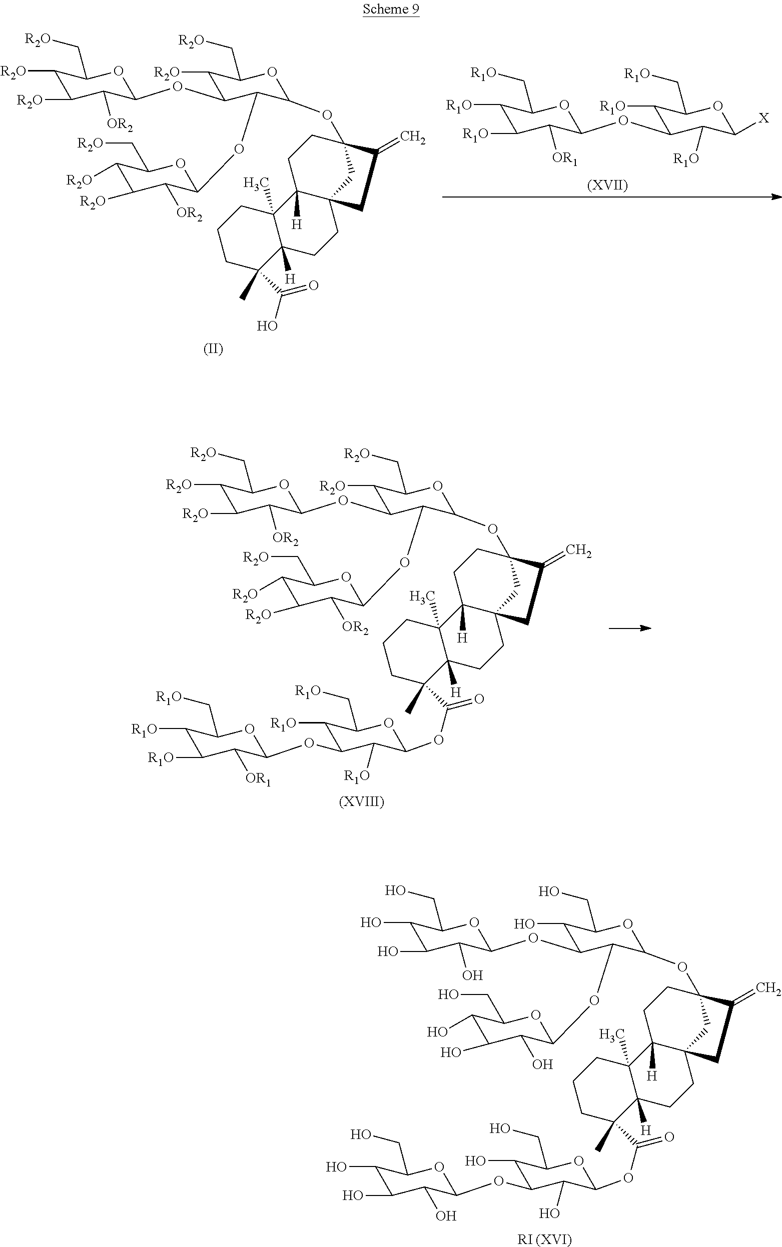

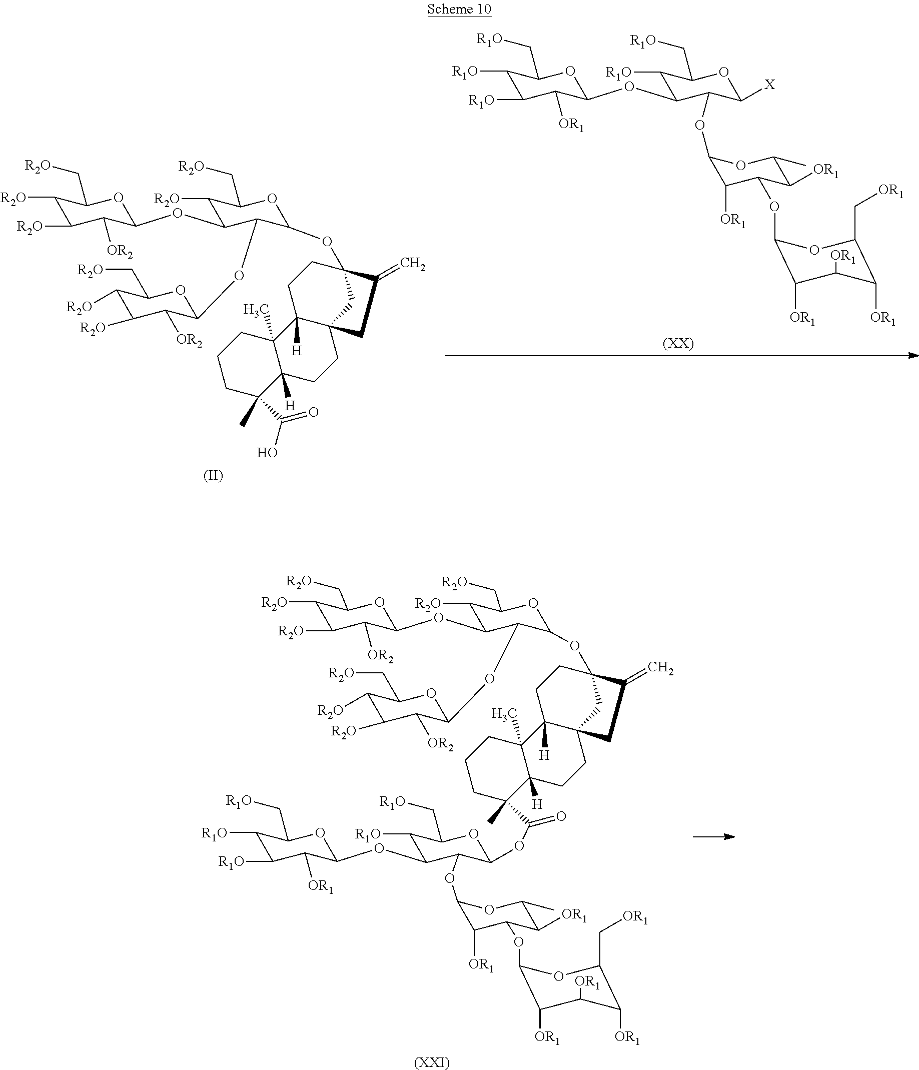

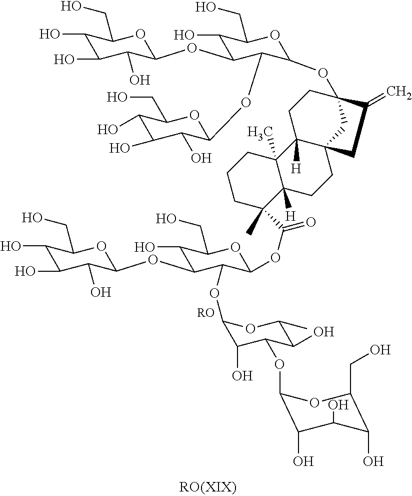

### ABBREVIATIONS

### ACN Acetonitrile

EtOAc Ethyl acetate
DMF Dimethyl formamide
PE Petroleum ether

### DCM Dichloromethane

### THF Tetrahydrofuran

HOAc Acetic acid
Ac₂O Acetic anhydride

### TEA Triethylamine

### DIPEA Diisopropylethylamine

### DMAP 4-Dimethylaminopyridine

### RD Rebaudioside D

### RA Rebaudioside A

### ST Stevioside

### RB Rebaudioside B

### RC Rebaudioside C

### RM Rebaudioside M

### RN Rebaudioside N

### RI Rebaudioside I

### RO Rebaudioside O

### NIS N-iodosuccinimide

### DBU 1,8-Diazabicycloundec-7-ene

TsOH p-Toluenesulfonic acid
TMSOTf Trifluoromethanesulfonic acid trimethylsilylester
TBSOTf Trifluoromethanesulfonic acid t-butyldimethylsilylester
TLC Thin layer chromatography
Aliquat336 Trioctylmethylammonium chloride
TBAC tetra-n-butylammonium chloride
TBAB Tetra-n-butylammonium bromide
TBAI Tetra-n-butylammonium iodide
TEBAc Benzyl triethyl ammonium chloride
PTC Phase transferring catalyst

### DIC N,N′-Diisopropylcarbodiimide

IDCP Iodonium dicollidine perchlorate

Certain specific aspects and embodiments of present invention are described in further detail by the examples below. The illustrated examples are not intended to limit the scope of this invention.

### EXAMPLES

### Example 1

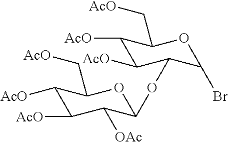 [SMILES: CC(=O)OCC1OC(Br)C(OC2OC(COC(C)=O)C(OC(C)=O)C(OC(C)=O)C2OC(C)=O)C(OC(C)=O)C1OC(C)=O]

### Preparation of (2R,3R,4S,5R,6S)-2-(acetoxymethyl)-6-((2R,3R,4S,5R,6R)-4,5-diacetoxy-6-(acetoxymethyl)-2-bromotetrahydro-2H-pyran-3-yloxy)tetrahydro-2H-pyran-3,4,5-triyl triacetate (Ia, α-acetobromosophorose)

The titled compound is prepared by lit. procedure on J.A.C.S. 78, 4709, 1957.

### Example 2

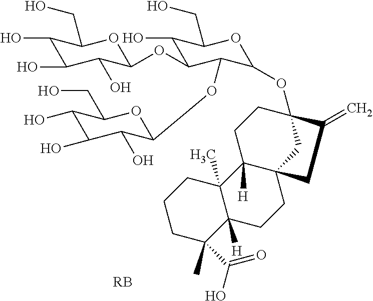

### Preparation of RB

A mixture of RA (15 g, 15.5 mmol) in aqueous KOH solution (30 mL, 5%) was stirred at 90° C. for 1 hour. It was cooled to 50° C., and then neutralized with 6 M aqueous HCl solution to pH 5. The solids were collected by filtration, washed with water and dried to afford Example 5 (RB, 12 g, 98%) as a white solid.

### Example 3

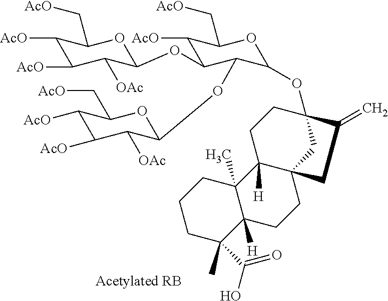

To a suspension of Example 2 (3.48 g, 4.3 mmol) in Ac₂O (6 mL, 63.6 mmol), were added DMAP (5 mg, 0.043 mmol) and triethylamine (2 mL, 12.9 mmol) subsequently. The reaction mixture was stirred at 60° C. for 2 hours. After cooled to room temperature, it was partitioned between dichloromethane and water. The organic extracts were concentrated to dryness to afford a residue. To a solution of this residue in MeOH (10 mL), was added 1% aqueous HCl solution (3 mL) dropwise with stirring. The mixture was stirred for 4 hours. It was neutralized with 2 M KOH to pH 4-5. The methanol was evaporated off in vacuo. The solids were collected by filtration to afford Example 6 (5.0 g, 95%) ¹H-NMR (CDCl3): δ 4.7-5.3 (10H), 0.9-2.0 (56H).

### Example 4

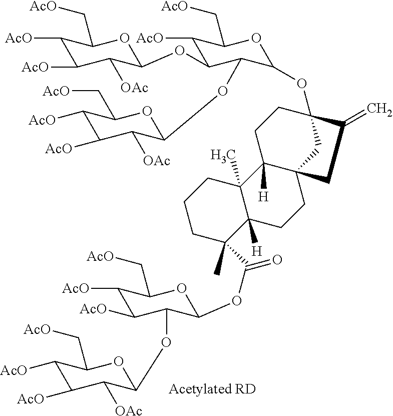

### Preparation of Acetylated RD:

### General Method:

The mixture of example 3 (1.5 g, 1 eq), appropriate base (Cs₂CO₃, or K₂CO₃, or K₂CO₃/KHCO₃ buffer, 8 eq), and PTC (TBAB, or TBAI, or Aliquat 336, 0.5 eq) in dichloromethane or 1,2-dichloroethane (7.5 mL) and water (7.5 mL) was heated at 40-65° C. To this, was added Example 1 (Ia′, 2.5 eq) over a period of 1.5 hrs. The resulting mixture was stirred at reflux for additional 1.5 hrs. The mixture was allowed to cool to room temperature, and the organic layer was isolated. The aqueous layer was extracted with appropriate solvent. The combined extracts were washed with brine, and dried over anhydrous Na₂SO₄. After filtration, the filtrate was concentrated to give the crude acetylated RD (yield: 25-54%). ¹H-NMR (CDCl₃): δ 5.6 (d, J=8.0 Hz, 1H), 3.5-5.4 (m, 36H), 1.9-2.2 (s, 51H, OC(O)CH₃), 0.8-1.9 (m, 26H).

### Example 5

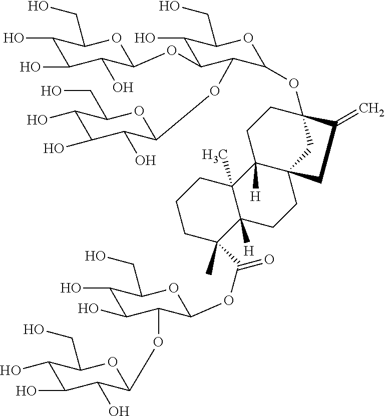

### Preparation of RD:

### Method A

To a solution of example 4 (2.0 g, 1.63 mmol) in MeOH (20 mL), was added MeONa/MeOH (105 mg, 25% wt, 0.3 eq). The reaction mixture was stirred at room temperature for 2 hrs. It was neutralized with 1 N HCl aqueous solution to pH 6. The solid was collected by filtration and re-crystallized in EtOH/water (2:1) to afford RD as a white solid (yield: 60-75%). ¹H-NMR (CD₃OD): δ5.6 (d, J=8.0 Hz, 1H), 5.3 (s, 1H), 4.7-5.0 (m, 5H), 3.0-4.0 (m, 29H), 0.9-2.4 (m, 26H).

Method B

To a solution of example 4 (2.0 g, 1.63 mmol) in MeOH (20 mL), was added K₂CO₃ (105 mg, 0.3 eq). The reaction mixture was stirred at room temperature for 2 hrs. The solid was removed by filtration. The filtrate was neutralized with 1N HCl aqueous solution to pH 6. The mixture was concentrated collected and re-crystallized in EtOH/water to afford RD as a white solid (yield: 50-70%).

### Example 6

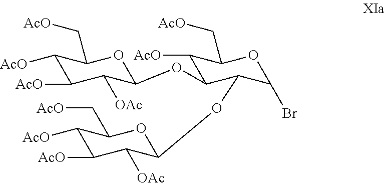 [SMILES: CC(=O)OCC1OC(Br)C(OC2OC(COC(C)=O)C(OC(C)=O)C(OC(C)=O)C2OC(C)=O)C(OC2OC(COC(C)=O)C(OC(C)=O)C(OC(C)=O)C2OC(C)=O)C1OC(C)=O]

### Preparation of XIa:

To a solution of acetylated RB (Example 3, 25.0 g, 20 mmol) in DCM (25 mL) at room temperature was added dropwise of HBr in acetic acid (33%, 12.5 mL) over a period of 30 min. After stirring for 3 hours, the reaction mixture was poured into a mixture of ice-water and DCM (50 mL). The organic layer was separated and washed with water, aqueous NaHCO₃ and brine subsequently, dried over anhydrous Na₂SO₄. After filtration, the filtrate was concentrated. The residue was purified by chromatography on silica gel eluting with 10% ethyl acetate in hexane to afford IXa (10.0 g, 50% yield).

### Example 7

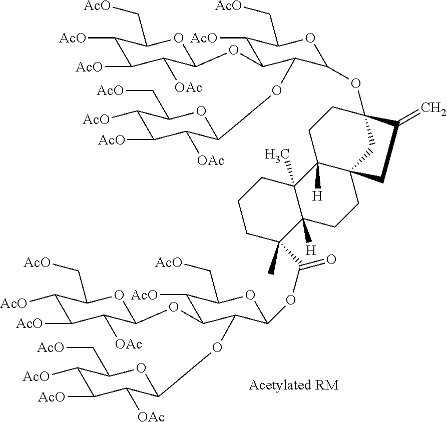

### Preparation of Compound XII (Acetylated RM):

To a solution of acetylated RB (Example 3, 62 g, 50 mmol) in THF (600 mL) and toluene (150 mL), was added water (300 mL) and followed by K₂CO₃ (49 g, 354 mmol) and TBAB (8.1 g, 25 mmol). The resulting mixture was heated to 50′. To this, IXa (Example 6, 100 g, 101 mmol) was added portionwise over 2 hours. After addition, the reaction mixture was stirred for further 2 hours. The mixture was allowed to cool to room temperature, and extracted with ethyl acetate. The combined extracts were washed with brine, dried over anhydrous Na₂SO₄ After filtration, the filtrate was concentrated. The residue was purified by chromatography on silica gel eluting with 10% 50% ethyl acetate in hexane to afford the titled product (57 g, 52% yield).

### Example 8

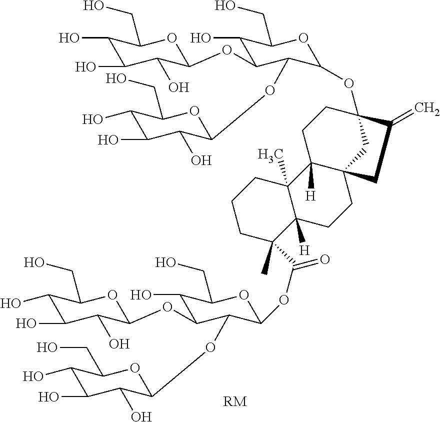

### Preparation of Compound X (RM):

To a solution of acetylated RM (Example 7, 30 g, 14 mmol) in MeOH (600 mL), was added K₂CO₃ (960 mg, 6.95 mmol). The reaction mixture was stirred at r.t. overnight. The mixture was neutralized with 1N HCl aqueous solution to pH 6. The mixture was concentrated collected and re-crystallized in EtOH/water to afford RM as a white solid (10.7 g, 58% yield). ¹H-NMR (CD₃OD): δ 5.46 (d, J=8.0 Hz, 1H), 5.07 (s, 1H), 4.78 (d, J=7.6 Hz, 1H), 4.69 (s, 1H), 4.64 (d, J=7.6 Hz, 1H), 4.47-4.52 (m, 3H), 2.86-4.0 (m, 36H), 0.78-2.19 (m, 26H).

1. A process for preparing a compound of formula (IV): 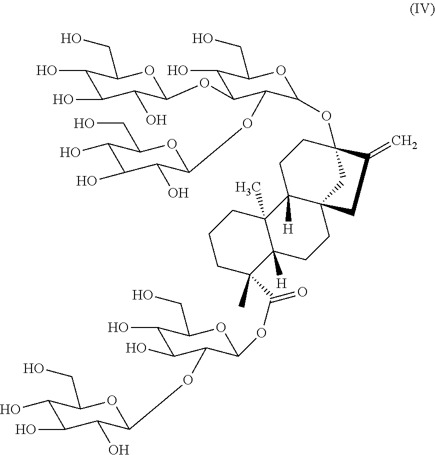
comprising:
(a) selectively hydrolyzing a compound of formula (VII), 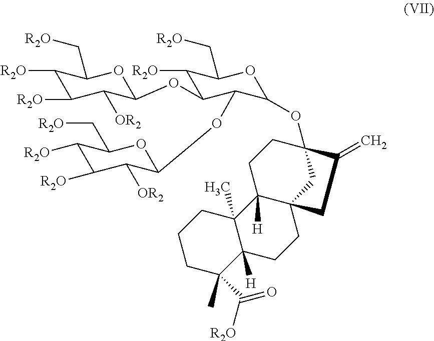
wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II), 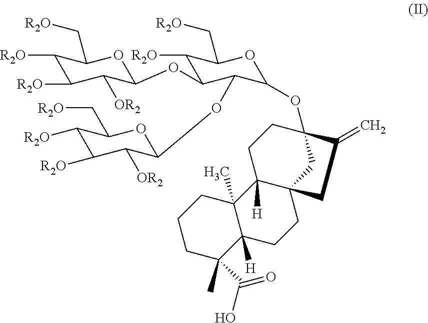
wherein each R₂ is defined as hereinabove;
(b) reacting the compound of formula (II) with a compound of formula (I), 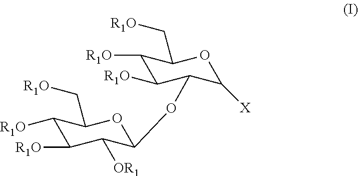
wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (III), 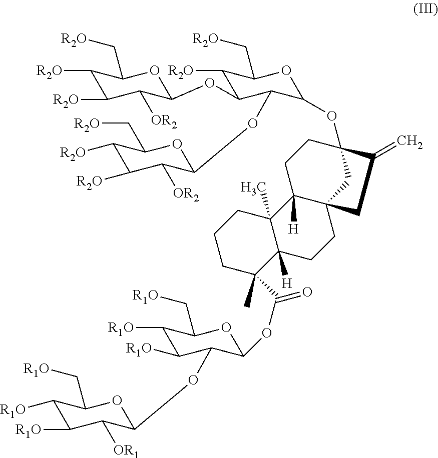
wherein R₁ and R₂ are defined as hereinabove;
(c) removing the protecting groups R₁ and R₂ of the compound of formula (III) to provide the compound of formula (IV).

2. A process for preparing a compound of formula (X): 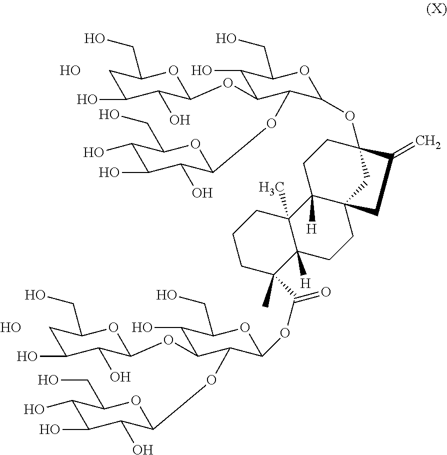
comprising:
(a) selectively hydrolyzing a compound of formula (VII), 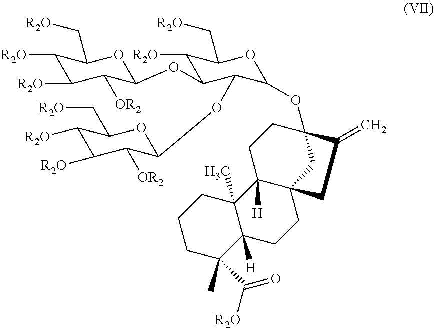
wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II), 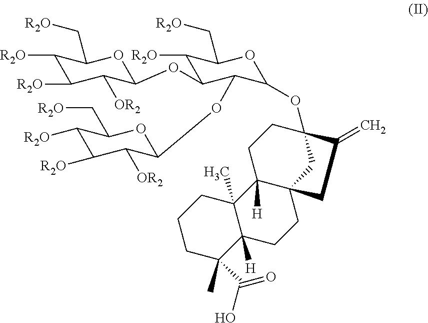
wherein each R₂ is defined as hereinabove;
(b) reacting the compound of formula (II) with a compound of formula (XI), 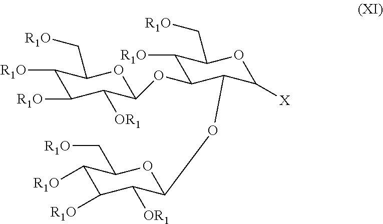
wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XII), 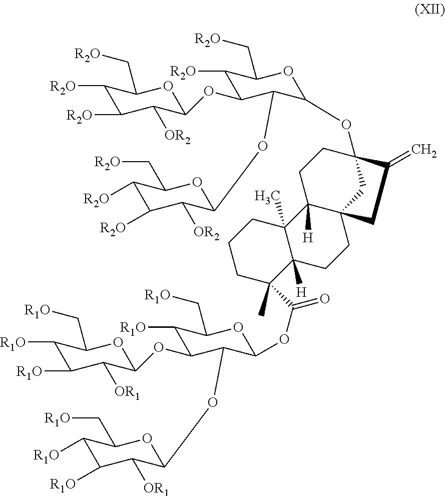
wherein R₁ and R₂ are defined as hereinabove;
(c) removing the protecting groups R₁ and R₂ of the compound of formula (III) to provide the compound of formula (X).

3. A process for preparing a compound of formula (XIII): 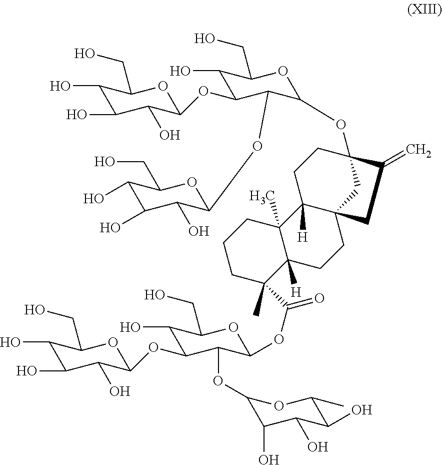
comprising:
(a) selectively hydrolyzing a compound of formula (VII), 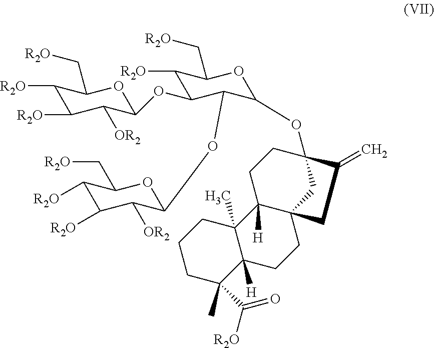
wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II), 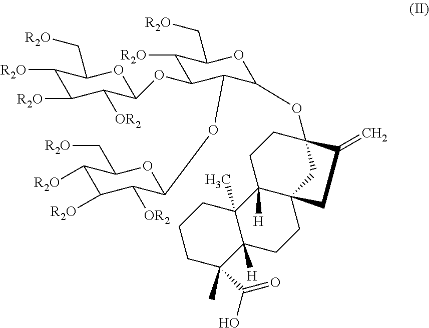
wherein each R₂ is defined as hereinabove;
(b) reacting the compound of formula (II) with a compound of formula (XIV), 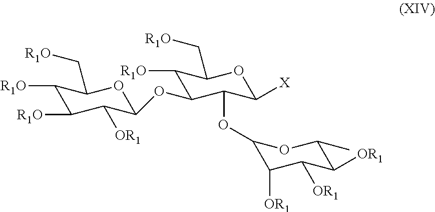
wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XV), 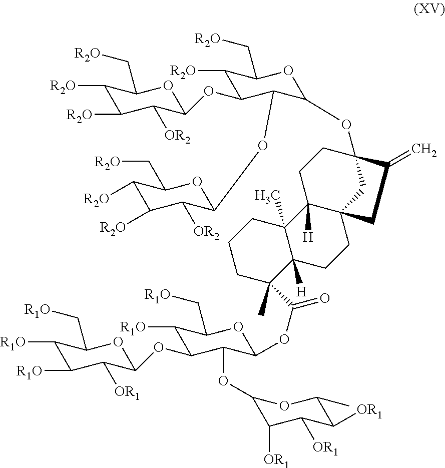
wherein R₁ and R₂ are defined as hereinabove;
(c) removing the protecting groups R₁ and R₂ of the compound of formula (III) to provide the compound of formula (XIII).

4. A process for preparing a compound of formula (XVI): 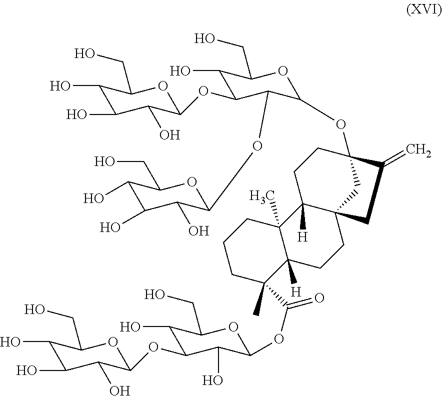
comprising:
(a) selectively hydrolyzing a compound of formula (VII), 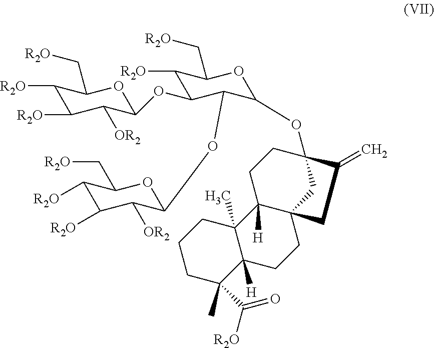
wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II), 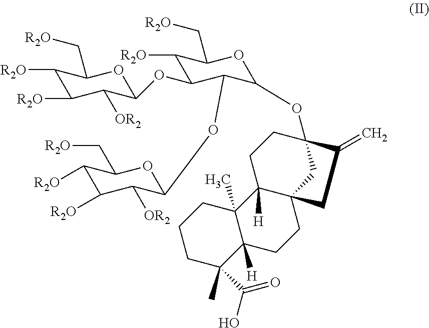
wherein each R₂ is defined as hereinabove;
(b) reacting the compound of formula (II) with a compound of formula (XVII), 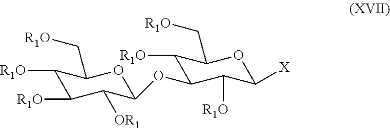
wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XVIII), 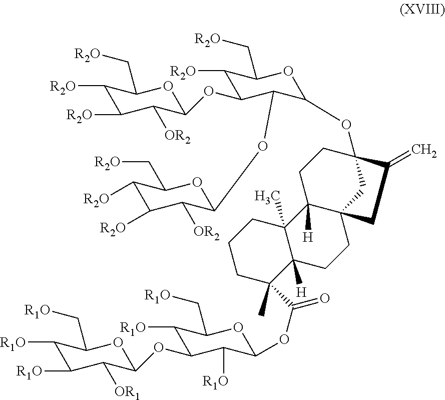
wherein R₁ and R₂ are defined as hereinabove;
(c) removing the protecting groups R₁ and R₂ of the compound of formula (XVIII) to provide the compound of formula (XVI).

5. A process for preparing a compound of formula (XIX): 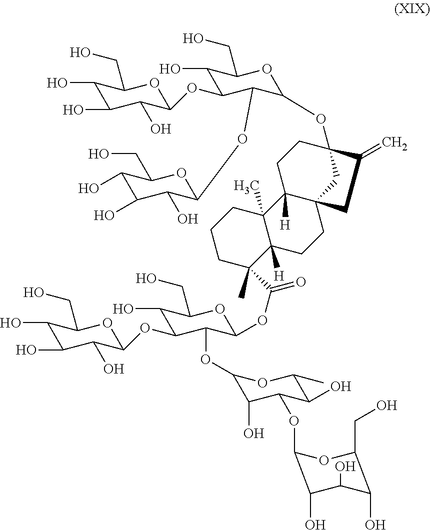
comprising:
(a) selectively hydrolyzing a compound of formula (VII), 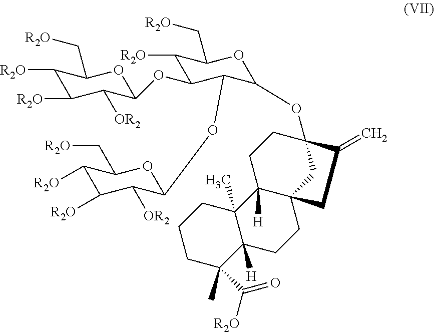
wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
to provide a compound of formula (II), 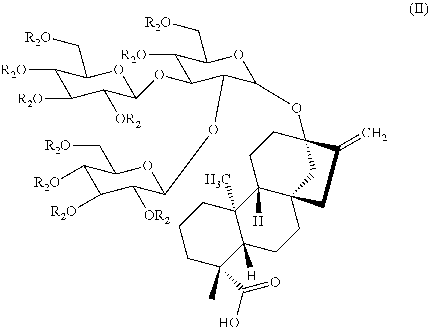
wherein each R₂ is defined as hereinabove;
(b) reacting the compound of formula (II) with a compound of formula (XX), 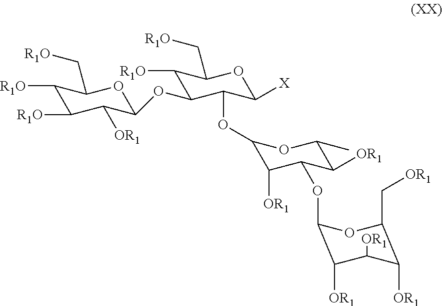
wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
X is halogen, OH, alkylthio, arylthio, sulfoxide (R′S(═O)—), sulfone (R′S(═O)₂—), sulfonate (e.g., mesylate, tosylate, or triflate), OC(═NH)CCl₃, OPO₃H, OPO₃R′ or OCH₂CH₂CH₂CH═CH₂, wherein R′ is (C₁-C₄)alkyl or aryl, and the anomeric carbon attached with X can be either α or β configuration;
to provide a compound of formula (XXI), 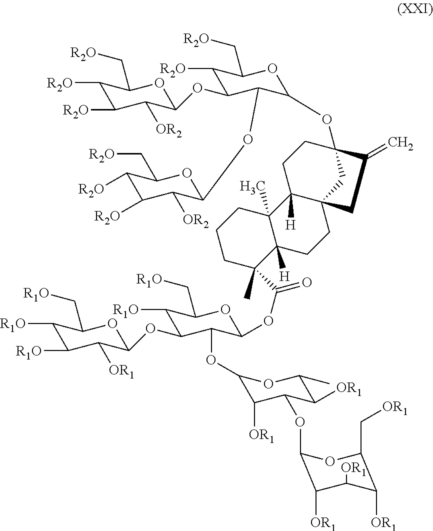
wherein R₁ and R₂ are defined as hereinabove;
(c) removing the protecting groups R₁ and R₂ of the compound of formula (III) to provide the compound of formula (XIX).

6. The process of any one of claims 1-5, wherein in step (b), the compound of formula (II) is reacted without the use of Ag₂CO₃.

7. The process of any one of claims 1-5, wherein each R₁ is independently (C₁-C₄)alkyl-acyl (i.e., (C₁-C₄)alkyl-C(═O)—) or phenyl.

8. The process of any one of claims 1-5, wherein each R₂ is independently (C₁-C₄)alkyl-acyl (i.e., (C₁-C₄)alkyl-C(═O)—) or phenyl.

9. The process of any one of claims 1-5, wherein each R₁ is independently acetyl (CH₃C(═O)—).

10. The process of any one of claims 1-5, wherein each R₂ is independently acetyl (CH₃C(═O)—).

11. The process of any one of claims 1-5, wherein the compound of formula (VII) is prepared by:
(a) hydrolyzing a compound of formula (VIII), 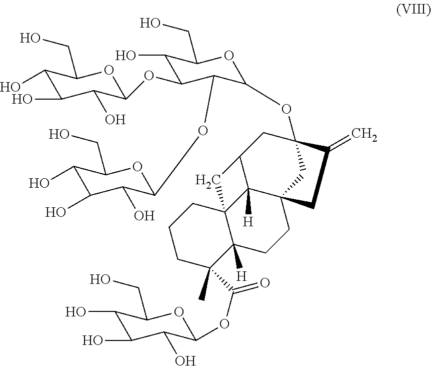
to provide a compound of formula (IX), 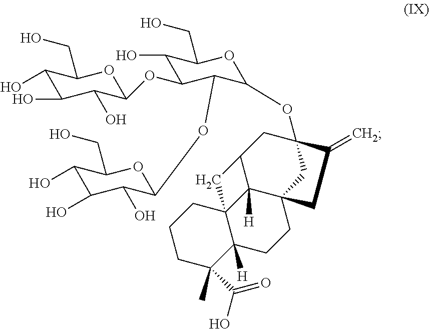
and
(b) protecting the OH groups of the compound of formula (IX) to provide the compound of formula (VII), 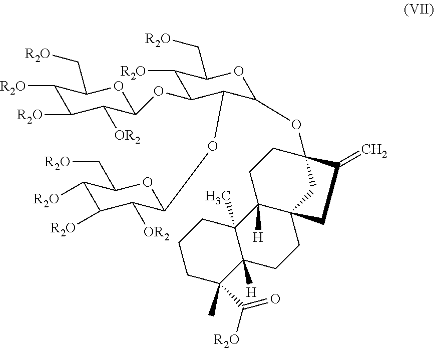
wherein R₂ is defined as hereinabove.

12. A process of preparing a compound of formula (III), 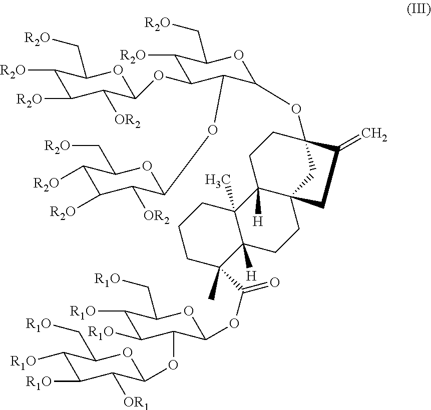
comprising reacting a compound of formula (II), 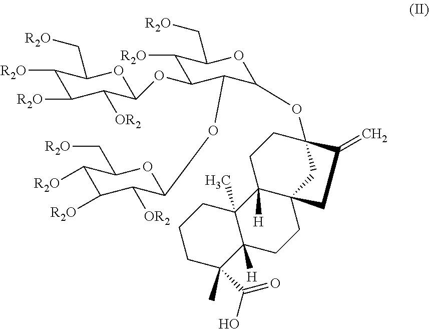
wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl;
(i) with a compound of formula (Ia) without the use of Ag₂CO₃, 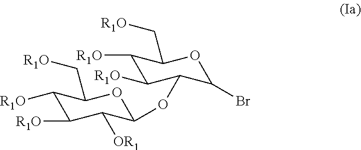
wherein each R₁ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl; and the anomeric carbon attached with Br can be either α or β configuration;
or (ii) with a compound of formula (Ib), 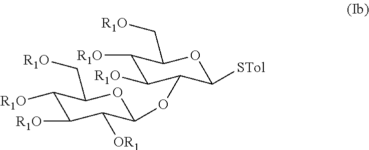
wherein each R₁ is as defined hereinabove;
or (iii) with a compound of formula (Id), 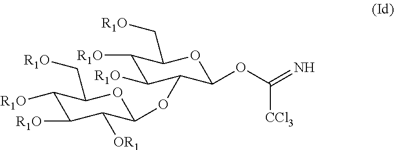
wherein each R₁ is as defined hereinabove;
to provide a compound of formula (III).

13. The process of claim 12, wherein the compound of formula (II) is reacted with the compound of formula (Ia) in the presence of a phase transferring reagent and an inorganic base.

14. The process of claim 13, wherein the phase transferring reagent is TBAB, TBAC, TBAI, or TEBAC; and the inorganic base is KHCO₃, K₂CO₃, K₃PO₄, or KH₂PO₄.

15. The process of claim 12, wherein the compound of formula (II) is reacted with the compound of formula (Ib) in the presence of NIS and an acid.

16. The process of claim 12, wherein the compound of formula (II) is reacted with the compound of formula (Id) in the presence of TBSOTf (CF₃SO₃SiMe₂t-Bu), TMSOTf, or TfOH.

17. The process of claim 12, further comprising removing the protecting groups R₁ and R₂ of the compound of formula (III) to provide a compound of formula (IV),

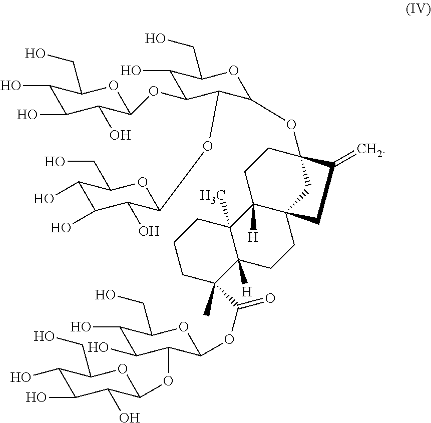

18. The process of claim 12, wherein in the compound of Formulae (Ia), (Ib) or (Id), the anomeric carbon attached with Br is α configuration.

19. The process of claim 12, wherein the compound of formula (II) is prepared by selectively hydrolyzing a compound of formula (VII), 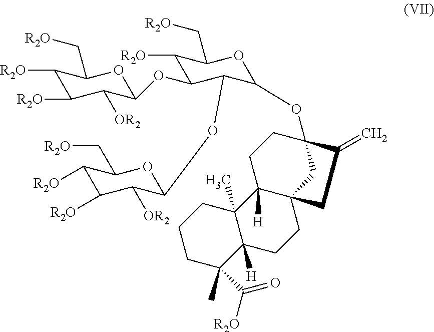
wherein each R₂ is independently a protecting group selected from benzyl, p-methoxybenzyl (PMB), benzyloxy methyl, p-methoxybenzyloxy methyl, (C₁-C₄)alkyl-acyl (e.g., acetyl), halogen substituted (C₁-C₄)alkyl-acyl (e.g., chloroacetyl, dichloroacetyl, trichloroacetyl, and trifluoroacetyl), aryl-acyl (e.g., benzoyl), trimethylsilylethoxymethyl (SEM), silyl protecting groups (e.g., trimethylsilyl, triethylsilyl, trisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, tris(trimethylsilyl)silyl, (2-hydroxystyryl)dimethylsilyl, 2-hydroxystyryl)disopropylsilyl, t-butylmethoxyphenylsilyl, t-butoxydiphenylsilyl), allyloxycarbonyl (alloc, —C(O)O—CH₂CH═CH₂), and t-butoxylmethyl.

20. The process of claim 19, wherein the compound of formula (VII) is prepared by:
(a) hydrolyzing a compound of formula (VIII), 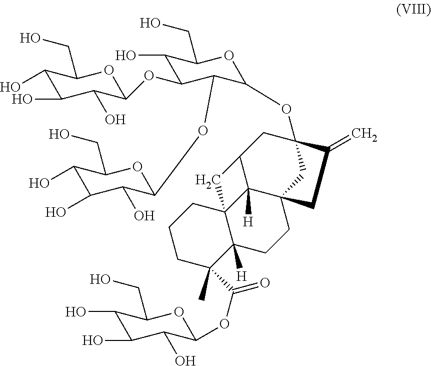
to provide a compound of formula (IX), 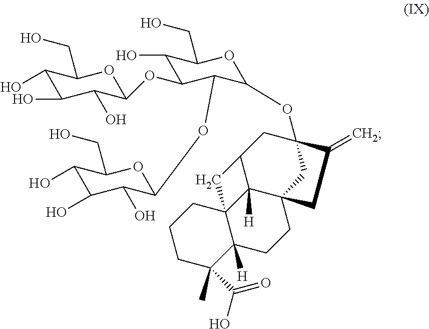
and
(b) protecting the OH groups of the compound of formula (IX) to provide the compound of formula (VII), 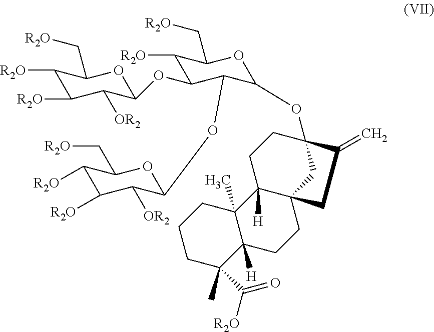
wherein R₂ is defined as hereinabove.

21. The process of claim 12, wherein each R₁ is independently (C₁-C₄)alkyl-acyl (i.e., (C₁-C₄)alkyl-C(═O)—) or phenyl.

22. The process of claim 12, wherein each R₂ is independently (C₁-C₄)alkyl-acyl (i.e., (C₁-C₄)alkyl-C(═O)—) or phenyl.

23. The process of claim 12, wherein each R₁ is independently acetyl (CH₃C(═O)—).

24. The process of claim 12, wherein each R₂ is independently acetyl (CH₃C(═O)—).
